(19) United States
(12) Patent Application Publication
(10) Pub. No.: US 2024/0165380 A1
(43) Pub. Date: May 23, 2024

(54) DEVICES, SYSTEMS, AND METHODS FOR AUTONOMOUSLY CONTROLLING A BALLOON CATHETER

(71) Applicants: Certus Critical Care, Inc., Salt Lake City, UT (US); Wake Forest University Health Sciences, Winston-Salem, NC (US)

(72) Inventors: Timothy WILLIAMS, Winston-Salem, NC (US); Daniel FONG, Sacramento, CA (US); Michael Austin JOHNSON, Holladay, UT (US); Lucas NEFF, Winston-Salem, NC (US)

(21) Appl. No.: 18/454,673

(22) Filed: Aug. 23, 2023

Related U.S. Application Data
(60) Provisional application No. 63/400,324, filed on Aug. 23, 2022.

(51) Int. Cl. A61M 25/10; A61M 5/172; A61M 25/00

(52) U.S. Cl. CPC A61M 25/10188; A61M 5/1723; A61M 2005/1726; A61M 2025/0002; A61M 2025/1052; A61M 2202/0415; A61M 2202/0427; A61M 2202/0429

(57) ABSTRACT

A method for automatically controlling an expandable member in a blood vessel of a subject may comprise obtaining, using a controller, proximal sensor data from a proximal pressure sensor and distal sensor data from a distal pressure sensor, comparing the obtained proximal sensor data to a proximal pressure threshold value, adjusting, using the controller, a volume of the expandable member based on the distal sensor data in response to determining that the proximal sensor data is above or at the proximal pressure threshold value, and adjusting, using the controller, the volume of the expandable member based on the proximal sensor data in response to determining that the proximal sensor data is below the proximal pressure threshold value.

### CROSS-REFERENCE TO RELATED APPLICATIONS

This application claims priority to U.S. Provisional Application No. 63/400,324, filed on Aug. 23, 2022, which is hereby incorporated by reference in its entirety.

### GOVERNMENT SUPPORT

This invention was made with government support under grant FA8650-20-2-6116 and W81XWH-21-C-0058 awarded by the United States Air Force/Air Force Material Command. The government has certain rights in the invention.

### TECHNICAL FIELD

This invention relates generally to the field of catheters for monitoring physiologic conditions. In particular, this invention relates to the field of autonomously controlling balloon catheters during a medical procedure.

### BACKGROUND

Over the years, there has been significant advancement in technologies to perform endovascular procedures. For example, endovascular catheters such as balloon catheters have been designed to be placed strategically in a blood vessel of a patient in order to partially and/or fully occlude a portion of the blood vessel. Balloon inflation may reduce blood pressure and blood flow downstream of the balloon (peripheral blood pressure), while augmenting blood pressure and increasing blood flow to branch vessels upstream of the balloon (e.g., central blood pressure).

Recently, advances have been made to automatically control balloon catheters when placed in a patient's blood vessel. However, existing automated technologies do not sufficiently ensure patient safety. For example, during endovascular procedures, the patient's condition can become unstable due to blood loss caused by trauma (e.g., vascular trauma). The patient's blood pressure proximal to the balloon (e.g., above blood pressure) and closest to the heart may drop to dangerous levels, thereby affecting one or more organs such as, for example, the brain, lungs, heart, etc. of the patient. It may be possible to avoid or minimize the adverse effect on the patient by adjusting the volume of the balloon. Additionally, the patient may require blood products (e.g., blood transfusion), intravenous medications, intravenous fluids, etc. to stabilize his or her condition. Existing technologies are not equipped to track the hemodynamic condition of the patient and make real-time, automated adjustments to the balloon catheter or to devices to provide blood products or medications.

Accordingly, additional devices, systems, and methods are needed for autonomously controlling balloon catheters and medication, intravenous fluid, and blood delivery while ensuring patient safety.

### SUMMARY

In some variations, a method for automatically controlling an expandable member in a blood vessel of a subject may comprise obtaining proximal sensor data from a proximal pressure sensor and distal sensor data from a distal pressure sensor using a controller, comparing the obtained proximal sensor data to a proximal pressure threshold value, in response to determining that the proximal sensor data is above or at the proximal pressure threshold value, adjusting a volume of the expandable member based on the distal sensor data using a controller, and in response to determining that the proximal sensor data is below the proximal pressure threshold value, adjusting the volume of the expandable member based on the proximal sensor data using a controller.

In some variations, the proximal pressure threshold value may be a lower limit of a proximal pressure guard range and adjusting the volume of the expandable member based on the distal sensor data may further comprise in response to determining that the proximal sensor data is above an upper limit of the proximal pressure guard range: adjusting the volume of the expandable member such that the distal sensor data approaches an interim set point, and incrementing the interim set point a plurality of times using the controller. The interim set point may be lower than or at a target distal pressure value

In some variations, incrementing the interim set point for a subset of times of the plurality of times may comprise for each time of the subset of times incrementing the interim set point by a predetermined value after a set period of time. The predetermined value may be between 1 mmHg and 5 mmHg. The set period of time may be between 1 minute and 5 minutes.

In some variations, incrementing the interim set point may comprise: for each time of the plurality of times: comparing a difference between the distal sensor data and the interim set point to a lagging range using the controller, and in response to determining that the difference is within the lagging range, incrementing the set point by a predetermined value after a set period of time. The method may further comprise in response to determining that the difference is below the lagging range, maintaining the interim set point constant. The lagging range may be between 20 mmHg and 5 mmHg.

In some variations, the proximal pressure threshold value may be a lower limit of a proximal pressure guard range, and adjusting the volume of the expandable member based on the distal sensor data may further comprise: in response to determining that the proximal sensor data is within the proximal pressure guard range: holding an interim set point for the distal sensor data constant using the controller, and adjusting the volume of the expandable member such that the distal sensor data approaches the interim set point. The interim set point may be lower than or at a target distal pressure value.

In some variations, the proximal pressure threshold value may be a lower limit of a proximal pressure guard range. Adjusting the volume of the expandable member based on the proximal sensor data may further comprise adjusting the volume of the expandable member based on the proximal sensor data until the proximal sensor data is above a lower limit of the proximal guard range. In some variations, adjusting the volume of the expandable member based on the distal sensor data may further comprise adjusting the volume of the expandable member so that distal blood pressure obtained from distal sensor data reaches a target distal pressure. In some variations, the method may further comprise indicating whether the distal sensor data is progressing towards the target distal pressure value. In some variations, the indicating may further comprise: providing a first indicator if an interim set point is progressing towards the target distal pressure value within a predetermined first time period, and providing a second, different indicator if an interim set point remains constant or moves away from the target distal pressure value within the predetermined first time period. In some variations, providing the first indicator may include determining whether an interim set point during a prior time window is equal to the target distal pressure value and whether a difference between a current interim set point and the target distal pressure value is greater than or equal to an indicator value using the controller, and providing the second indicator includes determining whether the interim set point during the prior time window is equal to the target distal pressure value and whether the difference between the current interim set point and the target distal pressure value is less than the indicator value. In some variations, the prior time window may be one or more of the prior one minute, two minutes, three minutes, four minutes, or five minutes. In some variations, the indicator value may be −1 mmHg. In some variations, providing the first indicator may comprise displaying on a display a shape comprising a first color and providing the second indicator comprises displaying on the display the shape comprising a second color. In some variations, the method may further comprise providing a third indicator providing a progress of the distal sensor data towards the target pressure value during a prior time window. In some variations, providing the third indicator may comprise displaying a border around the shape. In some variations, the prior time window may be a time within the prior five minutes. In some variations, the method may further comprise providing a third indicator to denote that the distal sensor data is at a minimum distal value, and providing a fourth indicator to denote that a progress of the distal sensor data towards the target distal pressure value cannot be determined by the controller. The minimum distal value may correspond to the distal sensor data when the blood vessel is fully occluded.

In some variations, the method may further comprise in response to a user input to fully deflate the expandable member, adjusting the volume of the expandable member such that the proximal sensor data remains above the proximal pressure threshold value and the expandable membrane sensor reaches a target expandable member pressure. The target expandable member pressure may be a negative pressure. The method may further comprise maintaining a minimum proximal pressure. The minimum proximal pressure may be configured to support one or more of a brain, lung, or heart of the subject.

In some variations, a method for automatically controlling an expandable member in a blood vessel of a subject may comprise obtaining proximal sensor data from a proximal pressure sensor and distal sensor data from a distal pressure sensor using a controller, comparing the obtained proximal sensor data to a proximal pressure guard range, in response to determining that the proximal sensor data is above an upper limit of the pressure guard range, transitioning the controller to an adjust state. The controller may be configured to adjust a volume of the expandable member so as to move the distal sensor data towards a distal target value in the adjust state. The method may also comprise, in response to determining that the proximal sensor data is within the proximal pressure guard range, transitioning the controller to a hold state. The controller may be configured to not adjust a volume of the expandable member to maintain the distal sensor data in the hold state. The method may also comprise in response to determining that the proximal sensor data is below a lower limit of the pressure guard range, transitioning the controller to a proximal pressure control state.

In some variations, in the adjust state, the method may comprise adjusting, using the controller, a volume of the expandable member based on a variable distal interim set point. In some variations, in the adjust state, the method may further comprise adjusting, using the controller, the volume of the expandable member such that the distal sensor data reaches the variable distal interim set point. In some variations, in the adjust state, the variable distal interim set point may increase a plurality of times by a predetermined value.

In some variations, the method may further comprise detecting that the blood vessel is fully occluded, and after occlusion at a first time of the plurality of times, increasing the variable interim set point by a first predetermined value in the adjust state. In some variations, the first predetermined value may be 5 mmHg over the current distal blood pressure obtained at occlusion. In some variations, the method may further comprise, after a set period of time at a second time of the plurality of times, increasing the variable interim set point by a second, different predetermined value in the adjust state. In some variations, the second predetermined value may be 1 mmHg. In some variations, in the hold state, a volume of the expandable member may be adjusted based on a constant distal interim set point using the controller.

In some variations, in the proximal pressure control state, a volume of the expandable member is adjusted so as to move the proximal sensor data above the lower limit of the pressure guard range. In some variations, in the proximal pressure control state a target value for the distal sensor data is set to a current value of the distal sensor data. In some variations, in response to a user input to fully deflate the expandable member, transitioning the controller to a wean to off state. The controller may be configured to fully delate the expandable member in the wean to off state. In some variations, in the wean to off state, a volume of the expandable member may be adjusted such that the proximal sensor data remains above the lower limit of the pressure guard range.

In some variations, a system for controller blood flow may comprise an elongate body comprising an expandable member, a proximal pressure sensor, and a distal pressure sensor, a pump in fluid communication with the expandable member, and a controller communicatively coupled to the proximal pressure sensor, the distal pressure sensor, and the pump. The controller may be configured to obtain proximal sensor data from the proximal pressure sensor and distal sensor data from the distal pressure sensor, compare the obtained proximal sensor data to a proximal pressure threshold value, in response to determining that the proximal sensor data is above the proximal pressure threshold value, adjust a volume of the expandable member based on the distal sensor data, and in response to determining that the proximal sensor data is below the proximal pressure threshold value, adjust the volume of the expandable member based on the proximal sensor data.

In some variations, a system for controlling blood flow may comprise an elongate body comprising an expandable member, a proximal pressure sensor, and a distal pressure sensor, a pump in fluid communication with the expandable member, and a controller communicatively coupled to the proximal pressure sensor, the distal pressure sensor, and the pump. The controller may be configured to obtain proximal sensor data from the proximal pressure sensor and distal sensor data from the distal pressure sensor, compare the obtained proximal sensor data to a proximal pressure guard range, in response to determining that the proximal sensor data is above an upper limit of the proximal pressure guard range, transition the controller to an adjust state. The controller may be configured to adjust a volume of the expandable member so as to move the distal sensor data towards a distal target value in the adjust state. The controller may also be configured to in response to determining that the proximal sensor data is within the proximal pressure guard range, transition the controller to a hold state. The controller may be configured to maintain the distal sensor data in the hold state. The controller may also be configured to in response to determining that the proximal sensor data is below a lower limit of the proximal pressure guard range, transition the controller to a proximal pressure control state.

In some variations, the devices, systems, and methods described herein may include a pump and one or more controllers configured to execute a transfusion and deliver a blood product to a patient to help stabilize their condition. Exemplary blood products include without limitation, red blood cells, plasma, platelets, and cryoprecipitate. Other fluids, e.g., normal saline, Ringer's lactate, D5W, or medications may also be infused to assist in obtaining or maintaining hemodynamic stability.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a block diagram illustrating an exemplary variation of a system for measuring physiologic conditions.

FIG. 1B illustrates an exemplary variation of a system for measuring physiologic conditions.

FIG. 1C illustrates an exemplary variation of a system for measuring physiologic conditions.

FIGS. 2A and 2B illustrate exemplary variations of a user interface of a system for measuring physiologic conditions.

FIG. 3 illustrates another exemplary variation of a user interface of a system for measuring physiologic conditions.

FIG. 4 is a flowchart illustrating an exemplary variation of a method for autonomously controlling an expandable member.

FIGS. 5-10 provide exemplary sensor data and illustrate autonomous control of an expandable member based on the sensor data.

FIG. 11 illustrates an exemplary approach to estimating the volume of a blood product for delivery to a patient while hemorrhage is also being controlled.

FIG. 12 illustrates a fixed rate, fixed volume transfusion of a blood product.

### DETAILED DESCRIPTION

Non-limiting examples of various aspects and variations of the invention are described herein and illustrated in the accompanying drawings.

Systems, devices, and methods for autonomously or automatically controlling an expandable member (e.g., a balloon) in a blood vessel of a subject are described herein. More specifically, systems, devices, and methods for autonomously controlling an expandable member during an endovascular procedure while ensuring subject safety are described herein.

The terms “proximal” and “distal,” as used herein in relation to sensor(s) and/or particular localized blood pressure readings, refer to the physiology of the patient and/or blood flow directionality from the heart. For example, when discussing a patient's physiology, proximal blood pressure (e.g., cephalad) refers to the blood pressure closest to the heart and distal blood pressure (e.g., rostral) refer to the blood pressure farthest from the heart. The sensor configured to receive proximal blood pressure data is referred to as a “proximal sensor” and the sensor configured to receive distal blood pressure data is referred to as a “distal sensor” as further described herein. For instance, when an elongate body comprising an elongate member with a first sensor positioned at the tip of the elongate body (and on a first side of the expandable member) and a second sensor positioned at the base of the elongate body (and on a second opposite side of the expandable member) is inserted retrograde into the aorta from the common femoral artery, the sensor at the tip of the elongate body and closer to the heart may be referred to as the proximal sensor and the sensor at the base of the elongate body and furthest away from the heart may be referred to as the distal sensor. Similarly, when the elongate body is inserted from the brachial or axillary artery and projecting antegrade down the aorta, the sensor at the tip of the elongate body and further away from the heart may be referred to as the distal sensor and the sensor at the base of the elongate body and closer to the heart may be referred to as the proximal sensor.

During an endovascular procedure, a blood flow control device comprising an expandable member may be advanced into a blood vessel, such as, for example, the aorta, of a patient to affect blood flow in the blood vessel, and thereby blood pressure of the patient. In particular, the volume of the expandable member may be adjusted to control the blood flow in the blood vessel, and thereby the blood pressure of the patient. To partially and/or fully occlude the blood vessel, the volume of the expandable member may be increased such that the expandable member inflates or otherwise increases in size. This impedes the blood flow past the expandable member, increasing the patient's proximal blood pressure (blood pressure closest to the heart) while decreasing distal blood pressure (blood pressure furthest from the heart). In some variations, after an initial period of complete occlusion to allow the patient's proximal blood pressure to stabilize, the volume in the expandable member may be adjusted to a target distal blood pressure. This may allow blood to flow past the expandable member to perfuse one or more lower organs such as, for example, the liver, kidneys, intestines, and/or the like while continuing to support the blood pressure above the balloon so that the brain, heart and lungs are perfused. Once the subject's physiologic conditions are fully stabilized, the expandable member may be removed from the blood vessel.

There are several complications that may arise when expandable members are used during endovascular procedures. For instance, in some situations, the expandable member may be overinflated. For example, the expandable member and/or the blood vessel may rupture, thereby adversely affecting the subject. Additionally, prolonged occlusion may lead to ischemia. Furthermore, in some situations, deflation of the expandable member may cause sudden physiologic derangements. For instance, deflation of the expandable member may cause the patient's proximal blood pressure to drop to dangerous levels, thereby adversely affecting the upper organs such as for example, the brain, heart, lungs, and/or the like. In such situations, the expandable member may have to be re-inflated to stabilize the physiologic conditions of the subject.

Systems, devices, and methods described herein provide for autonomous or automatic control of an expandable member based on information about the physiologic condition of the patient, thus assisting in preventing or minimizing the complications that may arise during expandable member-based endovascular procedures and increasing patient safety. The systems, devices, and methods described herein may also enhance the safety of expandable member-based endovascular procedures, such as those with the expandable member positioned within the aorta, by assisting in maintaining sufficient cephalad blood pressure to support the brain, lungs, heart, etc., while still allowing, when safely possible, blood flow to the extremities. Additionally, the devices, systems, and methods described herein allow for slow deflation of the expandable member, which may assist in avoiding sudden physiologic derangements. For example, slow deflation may provide time for the introduction of blood products, intravenous medications, intravenous fluids, and/or the like to stabilize the physiologic conditions of the subject. Furthermore, the devices, systems, and methods described herein provide feedback to a user (e.g., surgeon, operator, doctor, etc.). For example, a user may track the progress of the blood pressure of a patient towards a desired blood pressure value. For instance, the devices, systems, and methods described herein may allow the user to track the progress of the patient's distal blood pressure towards its target value. This allows the user to closely track the physiologic conditions of the subject so as to determine the underlying issues that may cause physiologic derangements. This may also allow the user to determine treatment decisions such as for example, when to deliver blood products, medications, and/or fluids, and the amount of blood products, medications, and/or fluids that may be needed. This may also allow the user to determine if the patient's physiology is permitting the device/system to track towards the target blood pressure, or if other interventions and or changes in the target blood pressure are needed.

The systems and devices described herein may comprise an elongate body comprising an expandable member. One or more pressure sensors may be positioned on the clongate body. For example, a first sensor may be positioned proximal to the expandable member and closer to a base of the elongate body a second sensor may be positioned distal to expandable member and closer to a tip of the elongate body. As described above, when the elongate body is advanced retrograde from the femoral artery into the aorta, the sensor closer to the tip of the elongate body may measure the patient's proximal blood pressure and may be referred to as the proximal sensor. In such a scenario, the sensor closer to the base of the elongate body may measure the patient's distal blood pressure may be referred to as the distal sensor. Similarly, when the elongate body is advanced antegrade into the aorta, such as through the brachial artery, the sensor closer to the tip of the elongate body may measure the patient's distal blood pressure and may be referred to as the distal sensor. In such a scenario, the sensor closer to the base of the elongate body may measure the patient's proximal blood pressure and may be referred to as the proximal sensor.

A pump may be in fluid communication with the expandable member. A controller may be communicatively coupled to the proximal pressure sensor, the distal pressure sensor, and the pump. The controller may automatically adjust a volume of the expandable member based on data from the proximal pressure sensor and the distal pressure sensor. For instance, to automatically adjust the volume of the expandable member, the controller may transition between a proximal pressure control state (e.g., state in which the patient's proximal pressure may be controlled) and a distal pressure control state (e.g., state in which the patient's distal blood pressure may be controlled). In the proximal pressure control state, the controller may adjust the volume of the expandable member based on data from the proximal pressure sensor and in the distal pressure control state, the controller may adjust the volume of the expandable member based on data from the distal pressure sensor.

The transition between the proximal pressure control state and the distal pressure control state may be based on a comparison of the proximal sensor data to a proximal threshold value. In some variations, the proximal pressure threshold value may be the minimum pressure value to which the proximal blood pressure may descend without adversely, or risk of adversely, affecting the subject. For example, if the proximal sensor data is equal to or above the proximal threshold value, the volume of the expandable member may be adjusted based on the distal sensor data. The controller may transition to the distal pressure control state when the proximal sensor data is equal to or above the proximal threshold value. In a similar manner, if the proximal sensor data is below the proximal threshold value, the volume of the expandable member may be adjusted based on the proximal sensor data. The controller may transition to the proximal pressure control state when the proximal sensor data is below the proximal threshold value.

As discussed above, the controller may transition to the proximal pressure control state when the proximal sensor data is below a minimum pressure value at which, or after which the subject may be adversely or at the risk of adversely being affected. Before the proximal pressure descends to the minimum pressure value (e.g., the proximal threshold value), the controller may be in the distal pressure control state. In the distal pressure control state, when the proximal sensor data is within a buffer amount from the proximal threshold value (i.e., above the proximal threshold value by a buffer amount), the controller may adjust the volume of the expandable member based on the distal pressure data such that the proximal sensor data may be prevented from descending below the proximal threshold value. This is called a hold state. However, when the proximal sensor data is above the buffer amount from the proximal threshold value, the controller may adjust the volume of the expandable member based on the distal pressure data such that the distal pressure data approaches a desired distal pressure value (e.g., a target distal value). This is called an “adjust state”.

Accordingly, in some variations, the proximal sensor data may be compared to a proximal guard range. The proximal threshold value may be a lower limit of the proximal guard range. The upper limit of the proximal guard range may be the proximal threshold value plus the buffer amount. If the proximal sensor data is above an upper limit of the proximal guard range, then the volume of the expandable member may be adjusted such that the distal sensor data approaches a variable interim set point. As discussed above, the controller may transition to the adjust state when the proximal sensor data is above an upper limit of the proximal guard range. If the proximal sensor data is within the proximal guard range (i.e., below the upper limit of the proximal guard range and above or at the proximal threshold value), then the volume of the expandable member may be adjusted such that the distal sensor data approaches a constant interim set point. Put differently, the interim set point may be held constant, and the volume of the expandable member may be adjusted such that the distal sensor data approaches the constant interim set point so as to stabilize the proximal pressure data and prevent the proximal pressure from descending below the proximal threshold value. As discussed above, the controller may transition to the hold state when the proximal sensor data is within the proximal guard range.

Adjusting the volume of the expandable member based on the distal sensor data (e.g., distal pressure control state) when the proximal sensor data is above or at the proximal threshold value allows for blood flow past the expandable member to perfuse the lower organs. Adjusting the volume of the expandable member based on the proximal sensor data when the proximal sensor data (e.g., proximal pressure control state) is below the proximal threshold value ensures that proximal blood pressure does not drop to dangerous levels, thereby ensuring perfusion of the heart and brain and the safety of the subject.

### SYSTEM

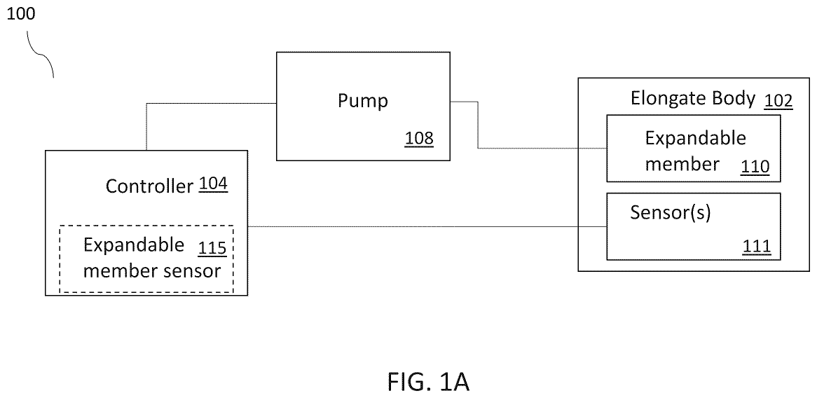

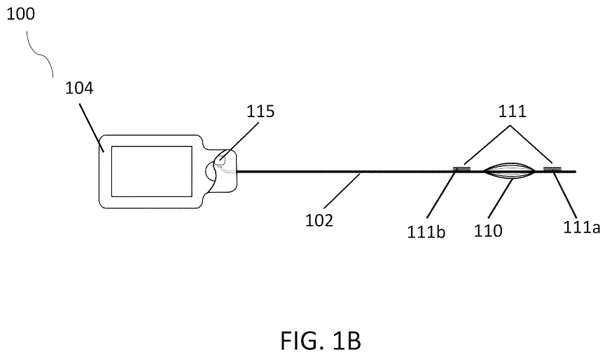

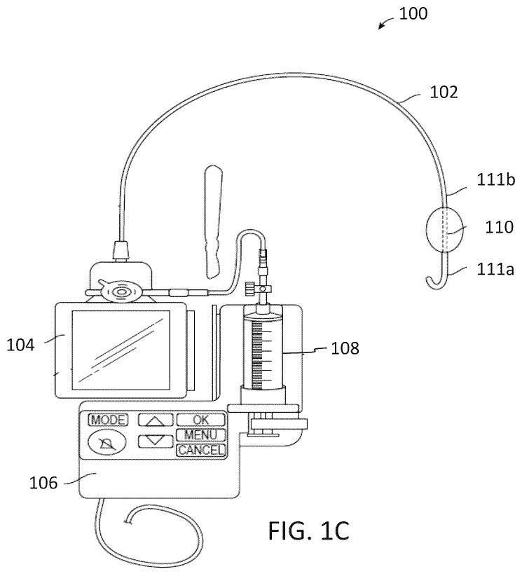

FIG. 1A is a block diagram illustrating an exemplary variation of a system 100 for autonomously controlling an expandable member. FIGS. 1B and 1C illustrate an exemplary variation of the system 100. The system 100 may include an elongate body 102 comprising an expandable member 110. In some variations, an expandable member sensor 115 configured to measure one or more characteristics of (e.g., pressure within) the expandable member 110 may be optionally positioned within a controller 104. For instance, the expandable member sensor 115 may be contained within, attached to, integrated with, or otherwise coupled to the controller 104. The expandable member 110 may be fluidly coupled via one or more fluid columns to the expandable member sensor 115. One or more sensors 111 may be coupled to the elongate body 102. For instance, one or more sensors 111 may be attached to, integrated with, and/or otherwise mounted on the elongate body 102. For example, the elongate body 102 may include a first sensor 111b closer to the base of the elongate body 102 and a second sensor 111a closer to the tip of the elongate body 102. As discussed above, the first sensor 111b and the second sensor 111a may be configured to receive localized blood pressure such as for example, proximal blood pressure and distal blood pressure. The terms “proximal” and “distal,” as used herein in relation to sensor(s) and/or particular localized blood pressure readings, refer to blood flow directionality from the heart. That is, “proximal” is closer to the heart while “distal” is further from the heart. This is not to be confused with the reversed usage of the terms when described from the perspective of a medical device such as a catheter, where the “distal end” of the medical device would commonly be understood as the end with the expandable element 110 furthest from the controller 104 and the “proximal end” would be understood as the end closer to the operator. For the purposes of illustration, the elongate body 102 in FIGS. 1A-1C is considered to be advanced retrograde in the aorta (i.e. insertion from the common femoral artery). Therefore, the sensor 111b is referred to as “distal sensor 111b” and sensor 111a is referred to as “proximal sensor 111a.” It should be readily understood that in instances when the elongate body is advanced from the brachial or axillary artery antegrade into the thoracic aorta, the sensor 111b may be referred to as “proximal sensor” and the sensor 111a may be referred to as the “distal sensor.”

In some variations, the system 100 may include more than one controller. For example, as seen in FIG. 1C, the system 100 may include a second controller 106 that may be operably coupled to the first controller 104. Controllers 104 and/or 106 may be communicatively coupled to the sensors 111 and/or the expandable member sensor 115 to receive and analyze sensor readings from the sensors 111 and/or the expandable member sensor 115. A pump 108 (e.g., a syringe pump) may be fluidly coupled to the expandable member 110 so as to adjust a volume of the expandable member 110. For example, the pump may be fluidly coupled via one or more fluid pathways (e.g., tubing, valves, lumen in the elongate body) to the expandable member 110 to inject fluid into and/or remove fluid from the expandable member 110.

### Elongate Body

The devices described herein may comprise an elongate body 102 comprising one or more sensors (e.g., one, two, three, four, five, or more), and in particular, the sensors may be integrated into the elongate body 102. The clongate body 102 may comprise a shaft sized and shaped for placement within a body of a patient (e.g., at a point of intervention such as in a blood vessel). In some variations, the elongate body 102 may be steerable. For example, in some variations, the elongate body 102 may be mechanically coupled to knobs, levers, pullwires, and/or the like that may be used to steer or otherwise deflect a distal end of the shaft of the elongate body 102. In some variations, the elongate body 102 may include one or more lumens therethrough. The lumen(s) may be partial lumen(s) (e.g., open on one end) and may be disposed within or lie within the movable shaft. The one or more lumens (e.g., two, three, four, or more) may serve any desired purpose. For example, in some variations, the lumens may be used for transmitting fluids to and from a patient's body and/or other components coupled to the clongate body, advancing and/or steering a guidewire into a desired location, housing other components (e.g., sensor wires, pressure sensing columns, imaging devices such as endoscopes, etc.) etc. In some variations, the lumen(s) may include an intake lumen and an exhaust lumen to deliver fluid and/or compressed gas through the elongate body. In some variations, the elongate body 102 may include a lumen to fluidly couple the expandable member 110 to the pump 108. The lumen fluidly coupling the expandable member 110 to the pump 108 may also serve as the fluid column fluidly coupling the expandable member 110 to the expandable member sensor 115. Additionally or alternatively, the elongate body may include another fluid column to fluidly couple the expandable member 110 to the expandable member sensor 115. The pressure within the expandable member 110 may be transduced via the fluid column to the expandable member sensor 115.

As mentioned above, the elongate body 102 may be sized and shaped for advancement to and placement at least partially within a target location of the patient's body. The elongate body 102 may be any diameter and length suitable for advancement to the target location. For example, the elongate body 102 may have a diameter between about 2 mm and about 36 mm. In some variations, the diameter may be for example between about 3 mm and about 25 mm, between about 4 mm and about 20 mm, or between about 5 mm and about 15 mm (including all values and sub-ranges therein). In some variations, the diameter may be for example between about 6 mm and about 10 mm. The elongate body 102 may have a length between about 1 cm and about 110 cm. In some variations, the length may be for example between about 10 cm and about 105 cm, between about 20 cm and about 100 cm, between about 30 cm and about 90 cm, between about 40 cm and about 80 cm, or between about 50 cm and 70 cm (including all values and sub-ranges therein).

In some variations, the elongate body 102 may comprise multiple layers. For example, one or more portions of the elongate body 102 may comprise a plurality of layers (e.g., two, three, four, or more), and all portions of the elongate body may comprise the same layers, or the layers may differ between different portions of the elongate body 102. In other variations, the elongate body 102 may comprise a single layer that may include one or more lumens therethrough. The elongate body 102, and/or any layer of the elongate body, may be formed from any suitable biocompatible material, such as, for example, Polytetrafluoroethylene (PTFE), polyimide, and Pebax®, a combination thereof, and the like.

Two or more sensors 111 (e.g., blood pressure sensors) may be integrated into the elongate body 102. One or more sensor wires associated with the sensors 111 may be routed through the elongate body 102. In some variations, the elongate body 102 may comprise an opening and/or a window to receive the sensor(s) 111 via a sensor housing. The window and/or opening may receive the sensor(s) 111 via the sensor housing as an inset. In some variations, the window and/or opening may be a cavity formed with an outer layer of the elongate body 102 that may receive the sensor(s) 111 and/or the sensor housing. In some variations, the sensor(s) 111 may be positioned on or otherwise may contact an outer surface of the elongate body 102. In some variations, the sensors 111 may be integrated into the elongate body 102 in a manner similar to that described in International Application No. PCT/US2022/049335, the content of which is hereby incorporated by reference in its entirety.

### Sensor(s)

As mentioned above, the elongate body 102 may include two or more sensor(s). In some variations, one or more sensor(s) on the elongate body may be sensor(s) 111. The sensor(s) 111 may be attached to, integrated with, and/or otherwise mounted on the elongate body 102 in any suitable manner. As discussed above, the sensors 111 may be integrated into the elongate body 102 similar to the integration described in International Application No. PCT/US2022/049335, the content of which is hereby incorporated by reference in its entirety.

The sensors 111 may be pressure sensors configured to measure changes in blood pressure. In some variations, in addition to the pressure sensors, the sensors 111 may include sensors configured to measure other physiologic conditions such as heart rate, respiratory rate, intracranial pressure, cerebral oxygenation, cerebral blood flow, electro-encephalographically, and the like. In some variations, the sensors 111 may include any sensor useful during a medical procedure such as, for example, temperature sensors, electrochemical sensors, impedance sensors, microelectrochemical system (MEMS) sensors, piezoelectric sensors, and/or the like. Any suitable number of sensors (e.g., one, two, three, four, or more) may be integrated into the elongate body to measure physiologic conditions.

In some embodiments, the devices may include two sensors, a first, distal sensor 111b, and a second, proximal sensor 111a, integrated into or otherwise coupled to the elongate body 102 (e.g., in instances when the elongate body 102 is advanced retrograde in the aorta (i.e. insertion from the common femoral artery). In some variations, the distal sensor 111b and/or the proximal sensor 111a may be placed at a distance between about 30 mm and about 10 mm, between about 25 mm and about 15 mm, between about 22 mm and about 18 mm from the expandable member 110. For instance, the distal sensor 111b and/or the proximal sensor 111a may be placed approximately 20 mm from the expandable member 110. In some variations, sensors 111 on the elongate body 102 may be situated at a specific distance from the ends of the expandable member 110 so as to acquire the physiologic data upstream and downstream of the expandable member 110.

Each of the distal sensor 111b and the proximal sensor 111a may measure patient physiologic information at a point of intervention to determine the patient's underlying physiology and to provide that information to a user. For example, in a variation in which the distal and proximal sensors 111b, 111a may be blood pressure sensors, the distal sensor 111b and the proximal sensor 111a may measure a local blood pressure of the patient at or around the position of the respective sensor. The data from the distal sensor 111b may be used to measure the distal systolic pressure and the distal diastolic pressure of the patient. For instance, distal systolic pressure and distal diastolic pressure may be derived from a waveform of the blood pressure. Distal systolic pressure may be measured by analyzing peaks of the waveform for a given time duration. Distal diastolic pressure may be measured by analyzing valleys of the waveform for the given time duration. In some variations, the data from the distal sensor 111b may be used to measure current distal proximal mean arterial pressure (DMAP). In some variations, DMAP may be the arithmetic mean of pressure samples received from the distal sensor 111b over a time window. In a similar manner, the data from the proximal sensor 111a may be used to measure the proximal systolic pressure and the proximal diastolic pressure of the patient. For instance, proximal systolic pressure and proximal diastolic pressure may be derived from a waveform of the blood pressure. Proximal systolic pressure may be measured by analyzing peaks of the waveform for a given time duration. Proximal diastolic pressure may be measured by analyzing valleys of the waveform for the given time duration. In some variations, the data from the proximal sensor 111a may be used to measure current proximal mean arterial pressure (PMAP). In some variations, PMAP may be the arithmetic mean of pressure samples received from the proximal sensor 111a over a time window.

The data from the sensor may be collected continuously or intermittently and may be collected over a defined period of time. In some variations, the data from the sensor may be collected continuously, such as for example, every 3 seconds, 4 seconds, 5 seconds, 6 seconds, 7 seconds, 8 seconds, 9 seconds, or 10 seconds (including all values and sub-ranges therein, such as, for example, between about 3 seconds and about 6 seconds, about 4 seconds and about 6 seconds, or between about 5 seconds or about 6 seconds). In some variations, the data from the sensor 118 may be collected every 5 seconds at 200 Hz.

### Expandable Member

The expandable member 110 may be disposed on, coupled to, integrated with, attached to, and/or affixed to the shaft of the elongate body 102 and a size (e.g., volume) of the expandable member may be controllable by a controller 104 or a user. For example, the expandable member may be configured to expand and contract and/or inflate and deflate such that the size (e.g., volume) of the expandable member may change during use of the blood flow control system. In some variations, the expandable member may be an inflatable/deflatable balloon, while in other variations the expandable member may comprise a shape memory material. In yet other variations, the expandable member may be connected to a mechanical linkage (e.g., wires, etc.) to change the size of the expandable member. The expandable member 110 may comprise any suitable elastomeric material (e.g., polyurethane, silicone, etc.). Additionally or alternatively, the expandable member 110 may comprise polyester, nylon, etc.

Blood flow at a target location in a patient's body (e.g., target blood vessel) may be regulated or otherwise controlled by changing a size of the expandable member 110. Fluid and/or compressed gas may be delivered through one or more lumens in the elongate body 102 in order to control and/or adjust the size (e.g., volume) of the expandable member 110. In some variations, the expandable member 110 may be strategically placed within the aorta of a patient and the size of the expandable member 110 may control blood flow through the aorta of the patient such that blood flow proximal to the expandable member 110 may be impeded to augment blood pressure distal to expandable member 110. The outer surface of the expandable member 110 may be configured to contact or otherwise interface with the wall(s) of the patient's blood vessel (e.g., at complete occlusion). The expandable member 110 may have any suitable shape when inflated. In some variations, the expandable member 110 may have an oval cross-sectional shape along a longitudinal axis when inflated. In other variations, the expandable member 110 may have a spherical shape when inflated (e.g., may have a circular cross-sectional shape).

Although FIG. 1A illustrates a system 100 with a single expandable member 110, it should be readily understood that the elongate body 102 may include any number of suitable expandable members 110. For instance, the system 100 may include two, three, four, or more expandable members 110 disposed on, coupled to, integrated with, attached to, and/or affixed to the elongate body 102 in series. In variations comprising three or more expandable members, the distance between the expandable members may be the same, or it may be different. In some variations, the expandable members 110 may be balloons, which may be positioned in series along the length of the elongate body 102 or disposed within one another. In variations comprising a plurality of balloons, the balloons may be individually expanded and contracted, or they may be expanded and contracted together.

The expandable member 110 may be fluidly coupled to an expandable member sensor 115 that may be configured to detect a pressure inside the expandable member 110. For example, the expandable member 110 may be fluidly coupled to the expandable member sensor 110 via one or more fluid columns within the elongate body 102. The pressure inside the expandable member 110 may be transduced through the fluid columns to the expandable member sensor 115. In some variations, the controller 104 may include the expandable member sensor 115 and may be configured to analyze the pressure inside the expandable member 110 via sensor readings from the expandable member sensor 115.

### Pump

The system 100 may comprise a pump 108, such as a syringe pump, which may be operably (e.g., fluidly) coupled to the expandable member 110 to facilitate adjusting a size thereof. In some variations, the pump 108 may be contained within or otherwise carried by or coupled to the controller 104. Additionally or alternatively, the pump 108 may be communicably coupled to the controller 104. In some variations, the pump 108 may be operated manually (e.g., actuated by a user by hand, without use of the controller 104), or by using the controller 104 (e.g., by manually and/or automatically). In some variations, the pump 108 may be operated automatically or autonomously using the controller 104. Additionally or alternatively, the pump 108 may be operated via a user interface (e.g., buttons) on the controller 104. In some variations, the pump 108 may be detached from or otherwise decoupled from the controller 104, and may be operated manually so as to establish a position and initial level or volume of the expandable member 110.

The pump 108 may comprise or otherwise be coupled to the expandable member 110 comprising a lumen (e.g., tubing), which may in turn be coupled to a lumen of the elongate body 102 of the system 100. In this manner, the pump 108 may be in fluid communication with the expandable member 110.

In some variations, a set of one or more valves may be utilized to control the flow of a fluid, such as saline, and/or compressed gas, such as carbon dioxide. In some variations, the pump 108 may be fluidly coupled to a valve (e.g., a stopcock valve) that may regulate the flow of fluid and/or compressed gas to the expandable member 110.

The size (e.g., volume) of the expandable member 110 may be adjusted using the controller 104 and the pump 108. For example, the controller 104 may determine an amount of fluid and/or compressed gas that is to be injected into or removed from the expandable member 110 so as to adjust the size of the expandable member 110 and thereby affect blood flow. The controller 104 may control (e.g., move, modify, or control a position thereof) an actuator, which may be releasably coupled to the pump 108 (e.g., to an actuation element on the pump). The actuator may engage and move the actuation element, thereby moving a portion of the pump 108 such that the pump 108 may inject or remove the fluid and/or compressed gas from the expandable member 110 based on instructions from the controller 104. In some variations, removal of the fluid and/or compressed gas may be activated via a screw actuation. In some variations, the pump may be coupled to a position sensor, which may provide information on the position of a portion of the pump 108 and thus how much fluid has been delivered to the expandable member 110.

The pump 108 may be any suitable pump operably and/or communicatively coupled to an actuator so as to inject and/or remove the fluid and/or compressed gas from the expandable member 110. For example, the pump 108 may be a syringe pump, diaphragm pump, peristaltic pump, or other suitable pump.

In some instances, the syringe pump 108 may include one or more sensors configured to detect a position and/or movement of a component other than the plunger and may utilize that position and/or movement as a corollary for the position and/or movement of the plunger, or may otherwise utilize the position and/or movement of that component to inform a determination of the position and/or movement of the plunger, an amount of fluid transferred (delivered or received), and/or size of an expandable member 110. For example, in some variations, the pump 108 may comprise a sensor configured to detect a position and/or movement of the actuation mechanism, such as, for example, the motor of the pump 108. For example, the pump 108 may include optical sensors such as optical encoders and/or magnetic sensors such as magnetic encoders configured to detect a position and/or movement of the motor, which in turn may be used to determine movement and/or position of the plunger. In some variations, the sensors may be configured to detect a position of and/or track a movement of one or more other components of the pump 108 (e.g., gearbox, gears in the gearbox, an actuation element (e.g., a linear gear), an actuator (e.g., a circular gear, etc.)

### Expandable Member Sensor

The expandable member sensor 115 may be disposed on, affixed to, attached to, mounted on, coupled to, and/or otherwise contained within the controller 104. The expandable member sensor 115 may be configured to detect a characteristic of the expandable member 110 such as, for example, a pressure of fluid and/or compressed gas inside the expandable member 110. For example, the expandable member sensor 115 may be fluidly coupled to the expandable member 110 via one or more fluid columns (e.g., lumens within the elongate body, tubing)), such as, for example, through the elongate body 102. The pressure in the expandable member 110 may be transduced via the fluid pathways to the expandable member sensor 115 positioned and/or included in the controller 104. In some variations, the expandable member sensor 115 may measure the expandable member pressure. When the expandable member 110 is not distended, the expandable member pressure may be indicative of the pressure outside of and surrounding the expandable member 110. When the expandable member is inflated, the expandable member pressure may be indicative of an amount of inflation of the expandable member 110. The expandable member pressure may be indicative of the amount of inflation and deflation of the expandable member 110.

In some variations, data may be collected from the expandable member sensor 115 continuously such as, for example, every 3 milliseconds, 4 milliseconds, 5 milliseconds, 6 milliseconds, 7 milliseconds, 8 milliseconds, 9 milliseconds, or 10 milliseconds (including all values and sub-ranges therein, such as, for example, between about 3 milliseconds and about 6 milliseconds, about 4 milliseconds and about 6 milliseconds, or between about 5 milliseconds and about 6 milliseconds).

### Controller

The devices and/or systems described herein may comprise one or more controllers (e.g., controller 104 and controller 106). For example, the system 100 may comprise a first controller 104, which may be coupled to a base of the elongate body 102. The first controller 104 may be communicatively and/or operably coupled to the sensors 111 and/or the expandable member sensor 115. In some variations, a second controller 106 may be releasably coupled to the first controller 104 as shown in FIG. 1C. In such variations, the second controller 106 may be coupled to the elongate body 102 via the first controller 104. In some variations, the system 100 may not include the first controller 104 and the second controller 106 may be directly coupled to the elongate body 102. In some variations, the first controller 104 may be used primarily as a user interface (e.g., it may not provide a controlling capability for the system). In particular, the user interface of the first controller 104 may display physiological data related to the patient, data related to control state of the system, feedback to be provided to the operator, etc. Although the controller 104 may include a processor and/or may execute some functionalities that enable controlling the system, the controller 104 may itself not control the system. In some variations, the controller 104 and/or the controller 106, may be communicatively coupled to a separate controller contained in a separate device (e.g., a separate computing device such as smartphone, tablet, computer, or the like). In some variations, the separate device may be a handheld device. In variations in which a controller in a separate device is utilized, the separate device may run a software application that may be used to or may otherwise assist in controlling the system components as described herein.

The controller(s) (e.g., controller 104 and/or controller 106) may be communicatively coupled to the sensors, such as, for example, the sensors integrated into the elongate body and may receive data therefrom. The controller(s) may comprise a processor (e.g., CPU) that may process data and/or other signals to control one or more components of the system. The processor may be configured to receive, process, compile, compute, store, access, read, write, and/or transmit data and/or other signals. In some variations, the processor may be configured to access or receive data and/or other signals from one or more of a sensor and a storage medium (e.g., memory, flash drive, memory card). The processor may be configured to run and/or execute application processes and/or other modules, processes and/or functions associated with the device.

In some variations, data from the sensors may be analyzed at the controller(s) over a discrete period of time. For instance, the data may be analyzed for example, every 3 milliseconds, 4 milliseconds, 5 milliseconds, 6 milliseconds, 7 milliseconds, 8 milliseconds, 9 milliseconds, or 10 milliseconds (including all values and sub-ranges therein, such as, for example, between about 3 milliseconds and about 6 milliseconds, about 4 milliseconds and about 6 milliseconds, or between about 5 milliseconds and about 6 milliseconds).

In some variations, the controller(s) may be communicably coupled to a user interface. For example, the user interface may be a display on the controller(s). In some variations, the user interface may be a display on any suitable computing device (e.g., computer, smartphone, tablets, and the like) communicably coupled to the controller, via, e.g., the communication device or module described herein. The user interface may comprise an input device (e.g., touch screen) and output device (e.g., display device) and may be configured to receive data from the sensor. For example, the user interface may be configured to receive data from the sensor through secure communication protocols. In some variations in which the user interface is part of a computing device, the communication protocol may include entry of a device specific number and/or scanning of a machine-readable code (e.g., a barcode, a QR code, or the like) contained on or within any component of the system (e.g., controller, elongate body, pump) into or with an application contained thereon or accessed therefrom. In some variations, an input device may comprise a touch surface for an operator to provide input (e.g., finger contact to the touch surface) corresponding to a control signal. In some variations, a haptic device may be incorporated into one or more of the input and output devices to provide additional sensory output (e.g., force feedback) to the operator.

The system 100 described herein may be operated in three ways—1) the system 100 may be operated manually (e.g., by hand), 2) the system 100 may be operated by a controller (e.g., controller 104, controller 106, and/or a separate controller) in an automatic mode of operation, the system 100 may be operated by the controller 104 and/or controller 106 in a manual mode of operation.

In order to manually (e.g., hand operated) operate the system 100, a user (e.g., surgeon, doctor, operator, etc.) may manually operate the pump 108 (e.g., move a portion of the pump, such as, for example, a plunger of a syringe pump) to adjust the volume of the expandable member 110. When manually operating the system 100, the pump 108 may be decoupled from the controller 104 and/or controller 106.

Additionally, the controller 104 and/or controller 106 may be used to operate the system 100. The controller 104 and/or controller 106 may operate in two modes of operation - an automatic mode and a manual mode. In both of these modes, the pump 108 may be coupled to the controller 104 and/or controller 106.

In the automatic mode of operation, the controller 104 and/or controller 106 may receive one or more inputs (e.g., prior to the endovascular procedure and/or during the endovascular procedure) from a user indicating target physiologic conditions for the subject. The controller 104 and/or controller 106 may receive sensor data from the one or more sensors (e.g., proximal pressure sensor 111a and distal pressure sensor 111b). The controller 104 and/or controller 106 may automatically or autonomously adjust the volume of the expandable member 110 (e.g., by automatically controlling a movement of the pump 108) based on the sensor data and the target physiologic conditions as further described herein.

In the manual mode of operation, a user may utilize a user interface of the controller 104 and/or controller 106 to adjust the volume of the expandable member 110. For example, a user may press buttons included on a housing of the controller 104 and/or controller 106. Pressing the button may cause movement of the pump 108, thereby causing a change in the volume of the expandable member 110. For example, the user may press a first button to inject fluid out of the pump 108 and a second button to withdraw fluid into the pump 108.

The user may switch between the different operation modalities as desired. For example, during the endovascular procedure, the user may manually (e.g., hand-operated) operate the pump 108 initially during insertion of the elongate body 102 into the blood vessel and may manually inflate the expandable member 110, such as, for example, to occlusion. The user may then couple the pump 108 to the controller 104 and/or controller 106, and may switch between manual mode of operation using the controller 104 and/or controller 106 and automatic mode of operation using the controller 104 and/or controller 106. For instance, the user may choose to operate the system 100 by the controller 104 and/or controller 106 in the automatic mode of operation as further described herein. However, as the controller 104 and/or controller 106 automatically adjusts the volume of the expandable member, the user may decide to switch from the automatic mode of operation to the manual mode of operation using the controller 104 and/or controller 106.

In some variations, the controller 104 and/or controller 106 may only momentarily switch from the automatic mode of operation to the manual mode of operation upon receiving user input via the user interface, and then may immediately and automatically return to the automatic mode of operation after adjusting the volume of the expandable member as indicated by the user. In this manner, the user may momentarily “override” the automatic mode of operation of the controller by providing instructions to the controller to adjust the size (e.g., increase or decrease volume) of the expandable member via the user interface, after which the controller may remain in the automatic mode of operation without additional user action or instruction to re-enter the automatic mode.

In the automatic mode of operation, the controller 104 and/or controller 106 may be configured to automatically or autonomously control the expandable member as further described herein. For example, prior to or during an endovascular procedure, the controller 104 and/or controller 106 may be configured to receive one or more inputs from a user indicating a target blood pressure value. The controller 104 and/or controller 106 may be configured to obtain sensor data from the sensors (e.g., proximal pressure sensor 111a and distal pressure sensor 111b) and to automatically adjust the volume of the expandable member 110 such that the sensor data (e.g., proximal pressure data or distal pressure data) approaches and/or reaches the target blood pressure value. For example, the target blood pressure value may be a target distal pressure value and the controller 104 and/or controller 106 may be configured to automatically adjust the volume of the expandable member 110 such that the distal blood pressure (e.g., DMAP) based on distal sensor data obtained from the distal pressure sensor 111b approaches and/or reaches the target distal pressure value. However, as the distal blood pressure approaches the target distal pressure value, blood may flow past the expandable member 110 to perfuse the lower organs, and in some situations, the proximal blood pressure (e.g., PMAP) based on proximal sensor data obtained from the proximal pressure sensor 111a may begin to descend, or may descend, to an undesirable (e.g., unsafe) level. This may adversely affect, or may risk adversely affecting, the upper organs such as the brain, lungs, heart, etc. Therefore, in addition to being configured to adjust the volume of the expandable member 110 such that the distal blood pressure approaches the target distal pressure value, the controller 104 and/or controller 106 may be further configured to maintain the proximal blood pressure (e.g., PMAP) obtained from the proximal pressure sensor 111a above a threshold level so that a patient's proximal blood pressure does not descend to an undesirable level. It should be readily understood that the distal blood pressure may be determined from or otherwise based on distal sensor data obtained from the distal pressure sensor 111b. Similarly, proximal blood pressure may be determined from or otherwise based on proximal sensor data obtained from the proximal pressure sensor 111a.

To adjust the volume of the expandable member so that the distal blood pressure reaches the target distal value while the maintaining a patient's proximal blood pressure at or above a desired level, the controller 104 and/or controller 106 may be configured to transition between a proximal pressure control state and a distal pressure control state. In the proximal pressure control state, the controller may be configured to adjust the volume of the expandable member based on the proximal sensor data and in the distal pressure control state, the controller may be configured to adjust the volume of the expandable member based on the distal sensor data. For instance, the controller may be configured to compare the proximal sensor data to a proximal pressure threshold value. The controller 104 and/or controller 106 may be configured to transition to different states (e.g., the proximal pressure control state and the distal pressure control state) based on this comparison.

In some variations, the proximal pressure threshold value may be the minimum pressure value to which the proximal blood pressure may descend without adversely, or risk of adversely, affecting the subject. In some variations, the proximal pressure threshold value may be predetermined. For example, the user may determine the proximal pressure threshold value for the subject based on the subject's physiologic conditions. The controller 104 and/or controller 106 may be configured to receive the proximal pressure threshold value via, for example, the user interface, from the user before and/or during the endovascular procedure. In some variations, the proximal pressure threshold value may change during the course of an endovascular procedure, and the controller may be configured to receive a plurality of proximal pressure threshold values (e.g., a first value pre-procedure, a second value mid-procedure) and to change the proximal pressure threshold value based on, for example, user input. Additionally or alternatively, the proximal pressure threshold value may be determined by the controller 104 and/or the controller 106 based on the subject's physiologic conditions. In some variations, the proximal pressure threshold value may be a universal value that may be the same for all subjects, while in other variations, the proximal threshold value may be personalized for each subject.

### Distal Pressure Control State

As discussed above, the controller 104 and/or controller 106 may be configured to adjust the volume of the expandable member based on the data from the distal pressure sensor when the controller transitions to the distal pressure control state. The transition to the distal pressure control state may be made based on a comparison of the proximal sensor data to the proximal pressure threshold value. For instance, if the proximal blood pressure (e.g., PMAP) determined from the proximal sensor data is equal to or above the proximal pressure threshold value, the controller 104 and/or controller 106 may be configured to transition from a proximal pressure control state to a distal pressure control state. Proximal blood pressure remaining at or above the proximal pressure threshold value may indicate that blood flow to the brain, lungs, heart, etc. of the subject is sufficient enough to allow for increased blood flow to organs distal to the expandable member. Accordingly, in the distal pressure control state, the controller 104 and/or controller 106 may be configured to adjust the volume of the expandable member based on the distal sensor data (e.g., distal blood pressure such as DMAP). In some variations, in the distal pressure control state, the controller 104 and/or controller 106 may adjust the volume of the expandable member such that the distal sensor data may advance towards a target distal pressure value. For example, in some variations, the controller 104 and/or controller 106 may transition to a first state referred to as an adjust state in the distal control state. In the adjust state, the controller may be configured to adjust the volume of the expandable member such that the distal sensor data may advance towards the target distal pressure value.

As discussed herein, it may be advantageous to stabilize the physiologic conditions of the subject before the proximal blood pressure descends to an undesirable level, and thus, it may be advantageous to stabilize the physiologic conditions of the subject before the proximal blood pressure drops below the proximal pressure threshold value. Accordingly, in some variations, in the distal control state, the controller 104 and/or controller 106 may adjust the volume of the expandable member based on the distal sensor data such that the proximal blood pressure obtained from the proximal sensor data may be stabilized. For instance, in some variations, the controller 104 and/or controller 106 may transition to a second state referred to as a hold state in the distal control state. In the hold state, the controller 104 and/or controller 106 may be configured to adjust the volume of the expandable member such that the physiologic conditions of the subject (e.g., proximal blood pressure) may be stabilized before the proximal blood pressure descends to undesirable levels.

The controller 104 and/or the controller 106 may transition to the hold state and/or the adjust state based on a comparison of the proximal blood pressure to a proximal pressure guard range. When the proximal blood pressure is above the proximal pressure guard range, then the controller 104 and/or the controller 106 may be configured to transition to the adjust state. When the proximal blood pressure is within the proximal pressure guard range, then the controller 104 and/or the controller 106 may be configured to transition to the hold state.

The proximal pressure guard range may have an upper limit and a lower limit, thus forming the guard range therebetween. The lower limit of the proximal pressure guard range may be the proximal pressure threshold value described in more detail above. As discussed above, the upper limit of the proximal pressure guard range may be a value above the proximal pressure threshold value. For example, the upper limit of the proximal pressure guard range may be a predetermined amount of pressure value above the proximal pressure threshold value. Put differently, the upper limit of the proximal pressure guard range may be the proximal pressure threshold value plus a buffer amount. The buffer amount may be between about 1 mmHg and about 25 mmHg, between about 5 mmHg and about 50 mmHg, between about 5 mmHg and about 15 mmHg (including all values and sub-ranges therein). In some variations, the buffer amount may be about 5 mmHg.

As discussed above, in the adjust state, the controller 104 and/or controller 106 may be configured to adjust the volume of the expandable member 110 so that the distal blood pressure approaches and/or reaches the target distal pressure value. In the hold state, the controller 104 and/or controller 106 may adjust the volume of the expandable member 110 to stabilize the physiologic conditions of the subject as further described herein.

### Adjust State

In the adjust state, the controller 104 and/or controller 106 may be configured to advance the distal blood pressure towards a target distal pressure value. The controller 104 and/or controller 106 may be configured to transition to the adjust state when the proximal blood pressure is stable. For example, when the proximal blood pressure is above the upper limit of the proximal pressure guard range (i.e., proximal pressure threshold value plus the buffer amount discussed above), the controller 104 and/or controller 106 may be configured to transition to the adjust state (e.g., from a hold state and/or from a proximal pressure control state).

To advance the distal blood pressure towards a target distal pressure value, the controller 104 and/or controller 106 may set and/or determine an interim set point. The interim set point may be a blood pressure value that may be equal to or lesser than the target distal pressure value. The controller 104 and/or controller 106 may adjust the volume of the expandable member such that the distal blood pressure may approach the interim set point. Put differently, the interim set point may be an interim goal towards which the distal blood pressure may be advanced to so that the distal blood pressure may eventually approach the target distal pressure value. To advance the distal blood pressure towards the target distal pressure value, the interim set point may be varied or changed (e.g., incremented) throughout the adjust state.

As discussed above, the controller 104 and/or controller 106 may be configured to transition to the adjust state from a hold state and/or a proximal control state. However, in some variations, the controller 104 and/or controller 106 may be configured to transition to the adjust state immediately after initial occlusion. Put differently, the controller 104 and/or controller 106 may be configured to transition to the adjust state after the expandable member reaches occlusion for a first time. In such scenarios, the interim set point immediately after initial occlusion may be a predetermined value. For example, the interim set point immediately after initial occlusion may be 5 mmHg above the current occlusion distal blood pressure.

In some variations, when the controller 104 and/or controller 106 transitions to the adjust state, the controller 104 and/or controller 106 may be configured to first compare the initial distal blood pressure (e.g., current distal blood pressure at the point of transition to the adjust state) to the target distal pressure value. If the initial distal blood pressure is greater than or equal to the target distal pressure value, the controller 104 and/or controller 106 may be configured to set the interim set point to the target distal pressure value. In such a scenario, the interim set point may not be changed. The controller 104 and/or 106 may then be configured to adjust the volume of the expandable member such that the distal blood pressure approaches the interim set point. For example, the volume of the expandable member 110 may be adjusted in a manner that is intended to cause the distal blood pressure to approach the interim set point. For instance, the volume of the expandable member 110 may be adjusted rapidly to have the distal pressure reach the interim set point. The volume of the expandable member may be adjusted every 4 seconds, every 5 seconds, every 6 seconds, every 7 seconds, every 8 seconds, every 9 seconds, and/or every 10 seconds until the distal pressure reaches the interim goal.

However, if the initial distal blood pressure is less than the target distal pressure value, the controller 104 and/or controller 106 may be configured to vary the interim set point. For instance, the controller 104 and/or controller 106 may be configured to increment the interim set point multiple times in the adjust state. More specifically, the controller 104 and/or controller 106 may be configured to increase the interim set point multiple times in the adjust state. For example, in some variations, the controller 104 and/or controller 106 may be configured to increment the interim set point after a set period of time. In particular, in some variations, each increment may occur after a set period of time.

In other variations, however, the increment may not occur after the set period of time in the adjust state. For instance, in some instances in the adjust state, the controller 104 and/or controller 106 may be configured to maintain the interim set point without incrementing the interim set point after the set period of time. This may allow the distal blood pressure to approach closer to the interim set point if the distal blood pressure has been lagging behind the interim set point. This is further described in detail below.

As discussed above, the interim set point may be a pressure value that may be less than or equal to the target distal pressure value. The interim set point may be a stepwise function. More specifically, the controller 104 and/or controller 106 may increment the interim set point by a predetermined value after a set period of time. For example, the controller 106 may increment the interim set point by 1 mmHg every minute. In some variations, the predetermined value may be a percentage of a difference between the target distal pressure value and a current distal blood pressure. For example, if the current distal blood pressure is 10 mmHg and the target distal pressure value is 20 mmHg, the predetermined value may be a percentage of the difference between the target distal pressure value and the current distal pressure value, which is 10 mmHg in this example. For instance, the predetermined value may be about equal to or less than about 25% of the difference, about equal to or less than about 20% of the difference, about equal to or less than about 15% of the difference, about equal to or less than about 10% of the difference, about equal to or less than about 5% of the difference, or about equal to or less than about 1% of the difference (including all values and sub-ranges therein). In some variations, the predetermined value may be between about 1 mmHg and about 5 mmHg, between about 2 mmHg and about 4 mmHg, or between about 2.5 mmHg and about 3.5 mmHg (including all values and sub-ranges therein). In some variations, the set period of time may be between about 5 second and about 5 minutes, between about 30 seconds and about 4 minutes, or between about 1 minute and about 3 minutes (including all values and sub-ranges therein). Therefore, in the adjust state, the interim set point may be increased multiple times and the volume of the expandable member may be adjusted so that the distal blood pressure approaches the interim set point each time. For example, the volume of the expandable member may be adjusted every couple of seconds until the distal blood pressure (e.g., distal blood pressure at current time) reaches the interim set point (e.g., interim set point at the current time). Accordingly, the controller 104 and/or controller 106 may be configured to gradually move the distal blood pressure of the subject to the target distal pressure value in the adjust state.

In addition to gradually moving the distal blood pressure to the target distal pressure value in the adjust state, the controller 104 and/or controller 106 may monitor a progress of the distal blood pressure towards the interim set point. If the distal blood pressure is lagging behind the interim set point, the controller 104 and/or controller 106 may be configured to maintain the interim set point (i.e., hold the interim set point constant) until the distal blood pressure advances closer to the interim set point (referred to as “catch up operation”). However, if the distal blood pressure is not lagging behind the interim set point, then the controller 104 and/or controller 106 may be configured to increment the interim set point as described above. To determine whether or not the distal blood pressure is lagging behind the interim set point, the controller 104 and/or controller 106 may be configured to compare a difference between the distal blood pressure (e.g., at a current time) and the interim set point (e.g., at the current time) to a lagging range. The lagging range may be a range of pressure values that determines whether the distal blood pressure is lagging behind the interim set point. That is, if the difference between the distal blood pressure and the interim set point is within the lagging range, then the controller 104 and/or controller 106 may identify that the distal blood pressure is not lagging behind the interim set point. If, however, the difference between the distal blood pressure and the interim set point is below the lagging range, then the controller 104 and/or controller 106 may identify that the distal blood pressure is lagging behind the interim set point. If the difference between the distal blood pressure and the interim set point at that time is within the lagging range, the controller 104 and/or controller 106 may be configured to continue to change (e.g., increase) the interim set point by the predetermined value after the set period of time (e.g., increment the interim set point by 1 mmHg after 1 minute) as discussed above. However, if the difference between the distal blood pressure and the interim set point at the particular time is outside (e.g., below) the lagging range, the controller 104 and/or controller 106 may be configured to hold the interim set point constant. In some variations, the lagging range may be between about 5 mmHg and about 20 mmHg, between about 8 mmHg and about 18 mmHg, between about 10 mmHg and about 15 mmHg, or between about 12 mmHg and about 14 mmHg (including all values and sub-ranges therein). In some variations, the lagging range may be about 5 mmHg.

### Hold State

When the proximal blood pressure is within the proximal pressure guard range, the controller 104 and/or controller 106 may be configured to transition to the hold state. More specifically, as the proximal blood pressure drops below the upper limit of the proximal guard range while remaining above the proximal pressure threshold value (e.g., lower limit of the proximal guard range), the controller 104 and/or controller 106 may be configured to transition to the hold state. In some instances, when the proximal blood pressure descends below the upper limit of the proximal guard range, this may be an indication that the physiologic conditions of the subject are not stable. For example, it may be possible that the proximal blood pressure may subsequently further descend below the lower limit of the proximal guard range (e.g., proximal pressure threshold value), thereby potentially adversely affecting the subject. Therefore, it may be advantageous to stabilize the proximal blood pressure and the distal blood pressure of the subject before continuing to adjust the volume of the expandable manner to drive the distal blood pressure toward the target distal pressure value.

Accordingly, in the hold state, the controller 104 and/or controller 106 may be configured to adjust the volume of the expandable member such that the distal blood pressure may approach the interim set point, but may be configured to hold the interim set point constant. Holding the interim set point constant but continuing to advance the distal blood pressure toward the interim goal may allow the distal blood pressure to stabilize. Therefore, stabilizing the distal blood pressure may allow the proximal blood pressure to remain above the undesirable level.

### Proximal Pressure Control State

The controller 104 and/or controller 106 may be configured to adjust the volume of the expandable member based on the data from the proximal pressure sensor when the controller transitions to the proximal pressure control state. The transition to the proximal pressure control state may be made based on a comparison of the proximal sensor data to the proximal pressure threshold value. When the proximal blood pressure determined from the proximal sensor data (e.g., PMAP) descends below the proximal pressure threshold value, the controller 104 and/or controller 106 may be configured to transition to a proximal pressure control state. As discussed above, in some instances, the proximal blood pressure dropping below the proximal pressure threshold value may adversely affect, or may risk adversely affecting, the subject (e.g., the brain, lungs, heart, etc.). Therefore, in the proximal pressure control state, the controller 104 and/or controller 106 may be configured to adjust the volume of the expandable member 110 such that the proximal blood pressure ascends above the proximal threshold value, which may provide a sufficient and/or desired amount of blood flow to the brain, heart, and/or lungs of the subject.

As mentioned above, in the proximal pressure control state, controller 104 and/or controller 106 may be configured to adjust the volume of the expandable member based (e.g., solely) on the proximal blood pressure (e.g., to maintain the blood flow to the brain, heart, and/or lungs of the subject), Accordingly, the controller 104 and/or controller 106 may be configured to set the target distal pressure value to be a current value of the distal sensor data (e.g., current distal blood pressure). This target distal pressure value may be continuously reset as the volume of the expandable member is adjusted to reach the proximal pressure value. Because, the volume of the expandable member is adjusted solely based on proximal sensor data, the target distal pressure value may be any suitable value such as the current value of the distal sensor data. Accordingly, the controller 104 and/or controller 106 may set the interim set point for the distal sensor data to be the current value of the distal sensor data repeatedly as the current distal pressure value changes. The volume of the expandable member may be adjusted every couple of seconds so that the proximal blood pressure approaches proximal pressure threshold value.

### Wean to Off Control State

During the endovascular procedure and/or towards the end of the endovascular procedure (e.g., prior to removing the expandable member 110 from the subject's blood vessel), the user may choose to fully deflate the expandable member 110. For example, the user may indicate to the controller 104 and/or controller 106 that the user wishes to fully deflate the expandable member 110. In response to the indication from the user to fully deflate the expandable member 110, the controller 104 and/or controller 106 may be configured to transition to the wean to off control state.

In the wean to off control state, the controller 104 and/or controller 106 may be configured to adjust the volume of the expandable member such that distal blood pressure determined from the distal sensor data (e.g., DMAP) may advance to a target distal pressure. The target distal pressure value in the wean to off control state may be a pressure inside the expandable member 110 when the expandable member 110 completely deflated. For example, in some variations, the target expandable membrane pressure value may be a negative pressure.

In some variations, the target distal pressure value may be determined by the controller 104 and/or controller 106 (e.g., based on physiologic conditions of the subject). In some variations, the user may provide as input the target distal pressure value for the subject via a user interface (e.g., before starting the endovascular procedure and/or during the endovascular procedure). The controller 104 and/or controller 106 may adjust the volume of the expandable member 110 such that the distal sensor data may advance towards the target distal blood pressure. Unlike the distal pressure control state, in the wean to off control state, the controller 104 and/or controller 106 may be configured not to set any interim set point. Instead, the volume of the expandable member 110 may be adjusted such that the distal blood pressure is advanced towards the target distal blood pressure without any interim set points. Additionally or alternatively, the controller 104 and/or controller 106 may adjust the volume of the expandable member 110 such that the distal sensor data may advance toward an interim set point but no distal target setpoint is set. In this instance, the interim distal set point may be advanced until the expandable member pressure sensor obtains a negative number.

In some variations, the volume of the expandable member 110 may be adjusted faster in the wean to off control state than in the distal pressure control state. As an example, instead of adjusting the volume of the expandable member 110 to increment the distal blood pressure by a predetermined value every minute (e.g., such as in the distal pressure control state), the volume of the expandable member 110 may be adjusted to increment the distal pressure faster, such as for example, the volume of the expandable member 110 may be adjusted to increment the distal pressure by the predetermined value every 15 seconds. For instance, if the volume of the expandable member 110 may be adjusted to increment the distal blood pressure by 1 mmHg every minute in the distal pressure control state, then the volume of the expandable member 110 may be adjusted to increment the distal blood pressure by 1 mmHg every 15 seconds in the wean off control state.

In some variations, in the wean to off control state, the volume of the expandable member 110 may be adjusted faster when the proximal blood pressure is farther from the target proximal pressure value. However, as the proximal blood pressure advances towards the proximal target pressure value, the volume of the expandable member 110 may be adjusted so that the distal pressure advances towards the target distal pressure value at a slower pace. That is, the volume of the expandable member 110 may be adjusted every few seconds when the proximal blood pressure is farther from the target proximal value. As the proximal blood pressure approaches a range that may be closer to the target proximal value, the volume of the expandable member may be adjusted at a slower pace. This adjustment may become slower as the proximal blood pressure approaches a range that may be even closer to the target proximal value.

For example, the volume of the expandable member 110 may be adjusted such that the distal blood pressure is incremented by the predetermined value every 15 seconds when the proximal blood pressure is more than 20 mmHg away from the proximal target pressure value. But, the volume of the expandable member 110 may be adjusted such that the distal blood pressure may be incremented by the predetermined value every 30 seconds when the proximal blood pressure is between about 20 mmHg and 10 mmHg away from the proximal target pressure. The volume of the expandable member 110 may also be adjusted such that the distal blood pressure may be incremented by the predetermined value every minute when the proximal blood pressure is between about 10 mmHg and 5 mmHg away from the proximal target pressure. When the proximal blood pressure is within 5 mmHg of the proximal target pressure, the volume of the expandable member 110 may be adjusted to maintain the current interim distal pressure target without incrementing towards a higher distal pressure. The controller 104 and/or 106 may also transition from the wean to off mode to the proximal pressure control state when the proximal blood pressure is equal to or less than the proximal target pressure. It should be readily understood that the example described herein is solely for illustrative purposes. The pace at which the distal blood pressure and/or the proximal blood pressure is adjusted may be any suitable pace based on how far the proximal blood pressure is from the proximal target pressure.

In some variations, in the wean to off control state, the controller 104 and/ or controller 106 may transition from the wean to off control state to the proximal pressure control state if the proximal blood pressure approaches the proximal pressure threshold.

In this manner, in the wean to off control state, the volume of the expandable member 110 may be adjusted based on the proximal blood pressure if the proximal blood pressure descends below limits of the proximal guard range. In this scenario, the controller 104 and/or controller 106 may be configured to transition from the wean to off state to the proximal pressure control state. In this mode, the proximal pressure may serve as an input to expandable member titration. When the proximal blood pressure descends below the proximal pressure threshold value, the controller 104 and/or controller 106 may be configured to increase the volume of the expandable member in order to increase the proximal blood pressure to a pressure within the proximal guard range (i.e., above the proximal pressure threshold value).

### Delivery of Blood Products

Some variations of the devices and/or systems described herein may be configured to deliver a blood product (e.g., red blood cells, plasma, platelets) to the patient to stabilize their condition in addition to controlling hemorrhage. The blood product may be delivered with a blood product pump, which may be the same pump used to adjust the size of the expandable member, or may be a different pump than that used to adjust the size of the expandable member. When the blood product pump is different from the pump used to adjust the size of the expandable member, the blood product pump and expandable member pump may be disposed within the same housing (i.e., integrated into a single housing). In some variations, the blood product pump may be a syringe pump, a peristaltic pump, a pressure differential pump, or other pump designed for providing blood products. The blood product pump may be communicatively coupled to one or more controllers (e.g., controller 104, controller 106, or a different controller). One or more blood products may be delivered based on the hemodynamic condition or other physiologic condition of the patient.

The delivery of a blood product may occur via a manual mode of operation or an automatic mode of operation. When in a manual mode of operation, a user may utilize a user interface of the controller (e.g., controller 104, controller 106, or a different controller) to stop and/or start the infusion of the blood product. For example, a user may press one or more buttons included on a housing of the controller. Pressing a button may actuate a blood product pump, thereby causing a blood product to flow out of the pump.

When in the automatic mode of operation, the controller (e.g., controller 104, controller 106, or a different controller) may receive one or more inputs (e.g., prior to the endovascular procedure and/or during the endovascular procedure) from a user indicating target physiologic conditions for the patient. The controller may receive sensor data from the one or more sensors (e.g., proximal pressure sensor 111a and distal pressure sensor 111b). The controller may be configured to automatically or autonomously start and stop the infusion of the blood product based on the sensor data, target physiologic conditions, and/or one or more transfusion protocols, as further described below. A transfusion may be triggered by various conditions in which a blood pressure parameter (e.g., a proximal blood pressure, a distal blood pressure) is not being met while using the expandable member of the devices described herein to control hemorrhage, and in which transfusion of a blood product may be used to help maintain hemodynamic stability and perfusion of organs proximal and distal to the expandable member. The controller may also be configured to select a transfusion protocol based on whether various blood pressure parameters are met, as further described below. For example, the controller may select a transfusion protocol based on whether the distal blood pressure is meeting an interim set point within a predetermined period of time on the way to a target distal blood pressure value, where the proximal blood pressure or distal blood pressure values are falling while the expandable member volume remains constant over the predetermined period of time. In other examples, the controller may be configured to select a transfusion protocol based on whether the frequency of expandable member inflations to keep the proximal blood pressure within a proximal pressure guard range exceeds a threshold, or when proximal blood pressure continues to decline despite increases in expandable member volume with automated or manual inflations.

### Automated Transfusion: Fixed Rate, Fixed Volume (FRFV)

In some variations, the controller may utilize pressure data (e.g., proximal blood pressure, distal blood pressure, and/or expandable member pressure measurements) captured from a proximal sensor (e.g., a proximal blood pressure sensor), a distal sensor (e.g., a distal blood pressure sensor), and/or an expandable member sensor (e.g., a balloon pressure sensor) to trigger a fixed volume and/or a fixed rate of blood product delivery based on whether or not the distal blood pressure is making sufficient progress towards an interim set point or a target distal blood pressure value.

For example, when the controller is in a distal pressure control state, as described above, the expandable member volume (e.g., balloon volume) may be slowly decreased to increase the distal blood pressure to an interim set point. If the distal blood pressure fails to reach the interim set point within a predetermined time period, or the proximal blood pressure falls below the proximal pressure guard range (which may cause the interim set point to move away from the target distal blood pressure goal), then the controller may determine that the distal sensor data is not progressing towards the target distal blood pressure value. This lack of progression may be indicative of overall low blood volume within, or continued blood loss for, the patient. In some variations, the controller may determine that the distal sensor data is not progressing towards the target distal blood pressure value when the controller also determines that the expandable member has been inflated to an occluded state after having been previously deflated (e.g., in order to raise the distal pressure to the target distal pressure value). In these instances, the controller may initiate the delivery of one or more fixed (i.e., predetermined) volumes of a blood product at a fixed (i.e., predetermined) rate to the patient to restore sufficient blood volume and/or to provide additional hemodynamic support to the patient (e.g., to maintain sufficient perfusion and oxygenation of tissues).

These blood product transfusions may be delivered in volumes ranging from between about 5.0 ml to about 5,000 ml, including all values and sub-ranges therein. For example, the blood products may be delivered in volumes of about 5.0 ml, about 10 ml, about 20 ml, about 30 ml, about 40 ml, about 50 ml, about 60 ml, about 70 ml, about 80 ml, about 90 ml, about 100 ml, 150 ml, about 200 ml, about 250 ml, about 300 ml, about 350 ml, about 400 ml, about 450 ml, about 500 ml, about 550 ml, about 600 ml, about 650 ml, about 700 ml, about 750 ml, about 800 ml, about 850 ml, about 900 ml, about 950 ml, about 1,000 ml, about 1,500 ml, about 2,000 ml, about 2,500 ml, about 3,000 ml, about 3,500 ml, about 4,000 ml, about 4,500 ml, or about 5,000 ml.

In some instances, the blood product transfusions may be delivered in volumes ranging from about 100 ml to about 500 ml, including all values and sub-ranges therein. For example, the blood products may be delivered in volumes of about 100 ml, about 110 ml, about 120 ml, about 130 ml, about 140 ml, about 150 ml, about 160 ml, about 170 ml, about 180 ml, about 190 ml, about 200 ml, about 210 ml, about 220 ml, about 230 ml, about 240 ml, about 250 ml, about 260 ml, about 270 ml, about 280 ml, about 290 ml, about 300 ml, about 310 ml, about 320 ml, about 330 ml, about 340 ml, about 350 ml, about 360 ml, about 370 ml, about 380 ml, about 390 ml, about 400 ml, about 410 ml, about 420 ml, about 430 ml, about 440 ml, about 450 ml, about 460 ml, about 470 ml, about 480 ml, about 490 ml, or about 500 ml.

In other instances, the blood product transfusions may be delivered in volumes ranging from about 200 ml to about 400 ml, including all values and sub-ranges therein. For example, the blood products may be delivered in volumes of about 200 ml, about 205 ml, about 210 ml, about 215 ml, about 220 ml, about 225 ml, about 230 ml, about 235 ml, about 240 ml, about 245 ml, about 250 ml, about 255 ml, about 260 ml, about 265 ml, about 270 ml, about 270 ml, about 275 ml, about 280 ml, about 280 ml, about 285 ml, about 290 ml, about 295 ml, about 300 ml, about 305 ml, about 310 ml, about 315 ml, about 320 ml, about 325 ml, about 330 ml, about 335 ml, about 340 ml, about 345 ml, about 350 ml, about 355 ml, about 360 ml, about 365 ml, about 370 ml, about 375 ml, about 380 ml, about 385 ml, about 390 ml, about 395 ml, or about 400 ml.

Regarding rates of blood product transfusion, the blood products may be delivered at rates ranging between about 1.0 ml/min to about 2,500 ml/min, including all values and sub-ranges therein. For example, the blood products may be delivered at a rate of about 1.0 ml/min, about 2.0 ml/min, about 3.0 ml/min, 4.0 ml/min, about 5.0 ml/min, about 6.0 ml/min, about 7.0 ml/min, about 8.0 ml/min, about 9.0 ml/min, about 10 ml/min, about 11 ml/min, about 12 ml/min, about 13 ml/min, about 14 ml/min, about 15 ml/min, about 16 ml/min, about 17 ml/min, about 18 ml/min, about 19 ml/min, about 20 ml/min, about 25 ml/min, about 30 ml/min, about 35 ml/min, about 40 ml/min, about 45 ml/min, about 50 ml/min, about 55 ml/min, about 60 ml/min, about 65 ml/min, about 70 ml/min, about 75 ml/min, about 80 ml/min, about 85 ml/min, about 90 ml/min, about 100 ml/min, about 150 ml/min, about 200 ml/min, about 250 ml/min, about 300 ml/min, about 350 ml/min, about 400 ml/min, about 450 ml/min, about 500 ml/min, about 550 ml/min, about 600 ml/min, about 650 ml/min, about 700 ml/min, about 750 ml/min, about 800 ml/min, about 850 ml/min, about 900 ml/min, about 950 ml/min, about 1,000 ml/min, about 1,050 ml/min, about 1,100 ml/min, about 1,150 ml/min, about 1,200 ml/min, about 1,250 ml/min, about 1,300 ml/min, about 1,350 ml/min, about 1,400 ml/min, about 1,450 ml/min, about 1,500 ml/min, about 1,550 ml/min, about 1,600 ml/min, about 1,650 ml/min, about 1,700 ml/min, about 1,750 ml/min, about 1,800 ml/min, about 1,850 ml/min, about 1,900 ml/min, about 1,950 ml/min, about 2,000 ml/min, about 2,050 ml/min, about 2,100 ml/min, about 2,150 ml/min, about 2,200 ml/min, about 2,250 ml/min, about 2,300 ml/min, about 2,350 ml/min, about 2,400 ml/min, about 2,450 ml/min, or about 2,500 ml/min.

The blood product may be delivered until the proximal blood pressure is raised to a level where it is sufficiently above (e.g., greater than about 5.0 mmHg above) a proximal pressure guard value, which may provide time for the distal blood pressure to make sufficient progress towards the interim goal and target distal blood pressure value. One or more volumes of the blood product may be delivered to the patient. When a plurality of blood product volumes are delivered (e.g., one, two, three, four, etc.), the volumes may be transfused continuously (e.g., one volume after the other) or iteratively (e.g., separated by a predetermined amount of time). In some variations, the controller may continue to continuously or iteratively deliver volumes of the blood product until the controller determines that one or more threshold blood pressure values (e.g., a proximal blood pressure value, a distal blood pressure value) are met. The fixed volume nature of this blood product delivery may be useful in preventing waste of precious blood product resources while still restoring any blood loss of the patient.

### Automated Transfusion: Variable Rate, Variable Volume (VRVV)

In some variations, the automated delivery of blood products may be triggered by the same pressure data described above for fixed rate and/or fixed volume delivery (e.g., proximal blood pressure, distal blood pressure, and/or expandable member pressure measurements), but the blood products may be delivered at variable rates and/or in variable volumes. The determination by the controller of whether to proceed with a variable rate and/or variable volume delivery may be based on factors such as the rate of distal blood pressure decline and/or the frequency of expandable member inflations being made. The rate of distal blood pressure decline that may trigger the automated delivery of blood products according to a variable rate and/or variable volume protocol may range from about 1.0 mmHg/min to about 60 mmHg/min, including all values and sub-ranges therein. For example, the rate of distal blood pressure decline may be about 1.0 mmHg/min, about 5.0 mmHg/min, about 10 mmHg/min, about 15 mmHg/min, about 20 mmHg/min, about 25 mmHg/min, about 30 mmHg/min, about 35 mmHg/min, about 40 mmHg/min, about 45 mmHg/min, about 50 mmHg/min, about 55 mmHg/min, or about 60 mmHg/min. Regarding the frequency of expandable member inflations, inflation volumes over a six second time period may be used to determine whether to trigger the automated delivery of blood products according to a variable rate and/or variable volume protocol. In some variations, the volume may range from about 20 μl over a six second time period to about 5,000 μl over a six second time period, including all values and sub-ranges therein. For example, over a six second time period, the volume of expandable member inflation that may trigger the automated delivery of blood products according to a variable rate and/or variable volume protocol may be about 20 μl, about 50 μl, about 100 μl, about 150 μl, about 200 μl, about 250 μl, about 300 μl, about 350 μl, about 400 μl, about 450 μl, about 500 μl, about 550 μl, about 600 μl, about 650 μl, about 700 μl, about 750 μl, about 800 μl, about 850 μl, about 900 μl, about 950 μl, about 1,000 μl, about 1,100 μl, about 1,200 μl, about 1,300 μl, about 1,400 μl, about 1,500 μl, about 1,600 μl, about 1,700 μl, about 1,800 μl, about 1,900 μl, about 2,000 μl, about 2,100 μl, about 2,200 μl, about 2,300 μl, about 2,400 μl, about 2,500 μl, about 2,600 μl, about 2,700 μl, about 2,800 μl, about 2,99 μl, about 3,000 μl, about 3,100 μl, about 3,200, about 3,300 μl, about 3,400 μl, about 3,500 μl, about 3,600 μl, about 3,700 μl, about 3,800 μl, about 3,900 μl, about 4,000 μl, about 4,100 μl, about 4,200 μl, about 4,300 μl, about 4,400 μl, about 4,500 μl, about 4,600 μl, about 4,700 μl, about 4,800 μl, about 4,900 μl, or about 5,000 μl. In general, a variable rate and/or variable volume protocol may be employed when it is desired to quickly provide an amount of blood product to the patient.

In one variation, the controller may determine the rate and volume of the variable rate and/or variable volume blood delivery based on (e.g., by monitoring) distal blood pressure values. For example, because the magnitude of the distal blood pressure drop over a fixed time period may be proportional to the volume of blood loss (and thus, the volume of blood needed by the patient), and the slope of the distal blood pressure drop when no expandable member (e.g., balloon) adjustments are occurring may be proportional to the rate at which blood loss or overall patient decompensation is occurring, the controller may utilize the magnitude of the distal blood pressure drop over a fixed time period and/or the rate of change of the distal blood pressure drop when no expandable member adjustments are occurring (e.g., over a time period of about 2.0 seconds, about 3.0 seconds, about 4.0 seconds, about 5.0 seconds, or about 6.0 seconds) to determine the volume of blood product to be delivered and/or the rate of blood product delivery, respectively. As a more specific example, a drop in distal blood pressure of 10 mmHg over one minute may correspond to a loss of about 250 ml to about 500 ml in blood volume. The controller may determine that, in this instance, blood loss would be occurring at a rate of about 250 ml/min to about 500 ml/min (i.e., about 4.17 ml/sec to about 8.33 ml/sec) (Johnson MA, Davidson AJ, Russo RM, Ferencz SA, Gotlib O, Rasmussen TE, Neff LP, Williams TK. Small Changes, Big Effects: The Hemodynamics of Partial and Complete Aortic Occlusion to Inform Next Generation Resuscitation Techniques and Technologies. Journal of Trauma and Acute Care Surgery. 2017; 82(6): 1106-1111, the content of which is incorporated herein by reference in its entirety). Thus, if taking a conservative approach, and if the controller determined that the overall drop in distal blood pressure from about two minutes ago was about 15 mmHg and the current drop is 10 mmHg/min, then the controller may determine a blood transfusion volume of 750 ml be given at a rate of at 8.33 ml/sec or faster to restore the blood loss.

After each blood product transfusion is given, the controller may determine if the rate and/or volume of blood product delivery should be adjusted based on, for example, distal blood pressure measurements. For example, after each blood product transfusion is given, the controller may determine the rate of distal blood pressure decline and may compare this rate to a pre-infusion rate (e.g., a rate of distal blood pressure decline just before the prior blood product delivery). If the controller determines that the rate of distal blood pressure decline remains constant or is increasing. the controller may increase the rate and/or volume of blood product delivered. If the controller determines that the rate of distal blood pressure decline is decreasing, the controller may decrease the rate and/or volume of blood product delivered.

Additionally or alternatively, the controller may assess the magnitude of a patient's response to a blood product transfusion to predict a desired volume and/or rate of blood product delivery to reach one or more target blood pressure values, and may determine subsequently delivered blood product delivery rates and volumes based on the prediction(s). For example, the controller may utilize predictive analytics (e.g., a regression analysis) to determine a predicted volume and/or predicted rate to meet a desired hemodynamic goal (e.g., a target proximal blood pressure, a target distal blood pressure), and then may subsequently deliver the predicted volume of blood products and/or blood products at the predicted rate. This may be particularly useful in variations in which the rate of the distal blood pressure is recovering but has not yet recovered enough to reach a hemodynamic goal (e.g., proximal blood pressure, distal blood pressure). In this way, the controller may utilize an adaptive approach in determining the volume and/or rate of blood product delivery.

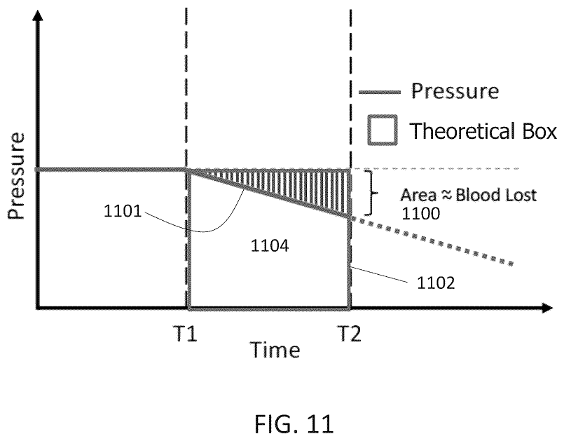

In further variations, the controller may utilize the distal blood pressure curve to estimate a volume of blood lost and may determine the volume of blood product to be delivered based on this estimation. For example, referring to FIG. 11, the controller may estimate the amount of blood volume lost based on an area (1100) above the distal blood pressure curve (1101) from the last time point in which the distal blood pressure was not declining (e.g., was constant) (T1), and where the upper limit that bounds the area (1100) is the last pressure where the distal blood pressure was not declining (e.g., was constant) to its current value (T2). In some instances, the controller may calculate the area (1100) above the curve (1101) by calculating the difference between the area of a theoretical box (1102) between T1 and T2 where the upper limit that bounds the theoretical box (1102) is the last pressure where the distal blood pressure was not declining (e.g., was constant) and the area (1104) of the distal blood pressure under the curve (1101). In other instances, the controller may determine a proportionality factor based on correlations between the area (1100) above the distal blood pressure curve (1101) and the volume of blood loss, and determine the volume of blood product to be delivered based on the proportionality factor. The proportionality factor may range from about 10 ml/mmHg per second to about 1,000 ml/mmHg per second, including all values and sub-ranges therein. For example, the proportionality factor may be about 10 ml/mmHg per second, about 20 ml/mmHg per second, about 30 ml/mmHg per second, about 40 ml/mmHg per second, about 50 ml/mmHg per second, about 60 ml/mmHg per second, about 70 ml/mmHg per second, about 80 ml/mmHg per second, about 90 ml/mmHg per second, about 100 ml/mmHg per second, about 150 ml/mmHg per second, about 200 ml/mmHg per second, about 300 ml/mmHg per second, about 350 ml/mmHg per second, about 400 ml/mmHg per second, about 450 ml/mmHg per second, about 500 ml/mmHg per second, about 550 ml/mmHg per second, about 600 ml/mmHg per second, about 650 ml/mmHg per second, about 700 ml/mmHg per second, about 750 ml/mmHg per second, about 800 ml/mmHg per second, about 850 ml/mmHg per second, about 900 ml/mmHg per second, about 950 ml/mmHg per second, or about 1,000 ml/mmHg per second. The analysis of data obtained from animal experiments may be used to determine the proportionality factor. For example, animal experiments in which parameters such as blood pressure and volume of blood lost are measured during a controlled hemorrhage may be performed and the data used to determine correlations therebetween to obtain the proportionality factor. Sections of the distal pressure blood pressure curve may be ignored where the expandable member volume is inflated (since this drop in distal blood pressure may not be due to blood loss) to reduce overestimating the amount of blood loss.

Additionally or alternatively, the controller may determine the rate and/or volume of blood product delivery based on the volume and/or frequency of expandable member (e.g., balloon) inflations (e.g., to maintain proximal blood pressure above a given proximal pressure guard value). This is because the volume and/or frequency of expandable member inflations may be proportional to the volume and/or rate of blood loss in the patient. For example, a larger expandable member inflation volume (such as, e.g., 1.0 ml as compared to 50 μl) may indicate a larger volume of blood loss and/or higher rate of blood loss. A high frequency of inflations (e.g., one or more inflations over a six second time period) may also indicate a larger volume of blood loss and/or higher rate of blood loss. In addition, the total increase in expandable member volume may be proportional to the overall amount of pressure support provided, and as such, may be proportional to the volume of blood product to be delivered. For example, a total increase in expandable member volume of about 1.0 ml during a 15 second time period may indicate that a larger blood product volume may need to be delivered.

In cases where expandable member inflation results in occlusion, rapid infusion of a blood product may be needed to help the patient survive. In these situations, the controller may be configured to deliver a predetermined amount of blood product at a predetermined rate. For example, in this situation, the controller may be configured to deliver a relatively, from a clinical perspective and considering size of the subject, large volume of a blood product, for example about 100 ml to about 7,000 ml (including all values and sub-ranges therein), at a relatively, from a clinical perspective and considering size of the subject, fast rate, for example, about 100 ml/min to about 250 ml/min (including all values and sub-ranges therein), rather than attempting to determine the rate and volume to provide. At occlusion, the expandable member may be unable to provide additional proximal pressure support (e.g., to maintain a target proximal pressure value that maintains perfusion of organs proximal to the expandable member such as the heart and brain) without potentially damaging the patient's blood vessel (e.g., aortic vessel), and in this instance, it may be beneficial for the controller to utilize blood product transfusions to hemodynamically support the patient.

### User Interface

In some variations, a user interface may be communicably coupled to the controller 104 and/or controller 106. In some variations, the user interface may be a display on the controller 104 and may be communicably coupled to the controller 106. Additionally or alternatively, the user interface may be a display on the controller 106 and may be communicably coupled to the controller 104. Additionally or alternatively, the user interface may be a display on any suitable computing device (e.g., computer, smartphone, tablets, etc.) communicably coupled to the controller 104 and/or controller 106.

In some variations, the user interface may comprise an input device (e.g., touch screen) and output device (e.g., display device) and be configured to receive input data from one or more of a user (or users), the controller 104 and/or controller 106, the pump 108, and the sensor(s) 111. For example, user control of an input device (e.g., keyboard, buttons, touch screen) may be received by the user interface and may then be processed by the controller 104 and/or controller 106 for the user interface to output a control signal to the controller 104 and/or controller 106 and/or the pump 108. Some variations of an input device may comprise at least one switch configured to generate a control signal. For example, an input device may comprise a touch surface or a user to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio data and recognize a user voice as a control signal.

A haptic device may be incorporated into one or more of the input and output devices to provide additional sensory output (e.g., force feedback) to the user. For example, a haptic device may generate a tactile response (e.g., vibration) to confirm user input to an input device (e.g., touch surface). As another example, haptic feedback may notify that user input is overridden by the pulsed electric field device.

In some variations, a user may input the proximal pressure threshold value, the target distal pressure value, the proximal pressure guard range, the predetermined value to increment the interim set point, the set period of time for incrementing the interim set point, the lagging range, etc. via the user interface. In some variations, the user may input an indication to transition the controller 104 and/or controller 106 to the wean off mode via the user interface.

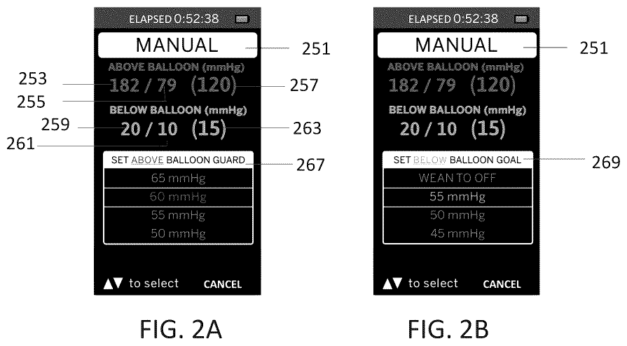

FIGS. 2A and 2B illustrate exemplary variations of user interfaces for inputting the proximal pressure threshold value and the target distal pressure value. As seen in FIGS. 2A and 2B, the user interface may display a mode of operation of the controller 104 and/or controller 106. In FIGS. 2A and 2B, the mode of operation may be a “manual” mode 251a. Additionally, the user interface may display the systolic proximal pressure 253, diastolic proximal pressure 255, PMAP 257, systolic distal pressure 259, diastolic distal pressure 261, and DMAP 263. In FIG. 2A, the user interface may enable the user to input the proximal pressure threshold value (e.g., 267). As an example, the user may choose 60 mmHg as the proximal pressure threshold value 267 for the subject. In FIG. 2B, the user interface may enable the user to input the target distal pressure value (e.g., 269). As an example, the user may choose a value (e.g., 50 mmHg) from a list of preloaded values as the target distal pressure value for the subject. In other variations, other input methods may be used, such as, for example, simply entering or otherwise providing a desired value using, e.g., a keypad, voice commands, a separate mobile device in communication with devices described herein, etc.

In some variations, the user interface may indicate whether the distal sensor data may be progressing towards the target distal pressure value. For example, through the endovascular procedure, the user interface may indicate whether the distal blood pressure may be progressing towards the target distal pressure value. The indication may be updated throughout the endovascular procedure. This may allow the user to determine the condition of the subject and/or to make treatment decisions.

The user interface may provide a status of progress towards the target distal pressure at a current point in time. For example, for every point in time, the controller 104 and/or controller 106 may be configured to determine the progress of the distal blood pressure towards the target distal pressure value. If the distal blood pressure is progressing towards the target distal pressure value, the user interface may display a first indicator. If the distal blood pressure is remaining constant and/or is moving away from the target distal pressure value, the user interface may display a second indicator. If the distal blood pressure is at a minimum value (e.g., distal blood pressure value that corresponds to distal blood pressure when the blood vessel is fully occluded), the user interface may display a third indicator. If the controller 104 and/or controller 106 does not have enough data to determine the progress of the distal blood pressure relative to the target distal pressure value, then the user interface may display a fourth indicator. In some variations, the progress at a current point in time may be displayed as a shape comprising a color. The shape may be any suitable shape such as, for example, a circle, a triangle, a square, a rectangle, an ellipse, and/or the like. As a non-limiting example, the first indicator may be displayed as a rectangle comprising a first color (e.g., green), the second indicator may be displayed as a rectangle comprising a second color (e.g., yellow), the third indicator may be displayed as a rectangle comprising a third color (e.g., red), and the fourth indicator may be displayed as a rectangle comprising a fourth color (e.g., black). While each indicator is described above as being displayed as the same shape (a rectangle), it should be appreciated that any shape and/or symbol may be used for each indicator, and the shapes and/or symbols may, but need not, be the same shape and/or symbols across the indicators.

In addition to displaying the progress at a current point in time, the user interface may also display the status of progress towards the target distal pressure at an immediately prior point in time. For instance, to determine whether the distal blood pressure is progressing towards a target blood pressure value, the controller 104 and/or controller 106 may be configured to determine whether an interim set point is progressing towards the target distal pressure value from an immediately prior point in time. More specifically, the interim set point progressing towards the target distal pressure value from an immediately prior point in time may in turn indicate that the distal blood pressure is progressing towards the target distal pressure value. This is because, as discussed above, the controller 104 and/or controller 106 may be configured to adjust the volume of the expandable member 110 such that the distal blood pressure may advance towards the interim set point.

Movement of the interim set point relative to the target distal pressure may assist the device and/or user in understanding the stability of the patient. For example, continued progress of the interim set point toward the target distal pressure may be reassuring as to the stability of the patient. In contrast, failure to increment the interim set point toward the target distal pressure due to a proximal pressure near or below the proximal pressure threshold value may indicate instability of the patient and failure to tolerate expandable member deflation/contraction. As discussed above, the user interface may display the status of progress towards the target distal pressure at a prior point in time. For example, the user interface may display the status of progress of the distal blood pressure ten minutes ago, nine minutes ago, eight minutes ago, seven minutes ago, six minutes ago, five minutes ago, four minutes ago, three minutes ago, two minutes ago, and/or the like. In some variations, the user interface may display the progress of the distal blood pressure five minutes ago. The progress at a prior point in time may be displayed as a border around the shape. For instance, if the progress at a current point in time is displayed as a shape (e.g., rectangle) comprising a color, the progress at a prior point in time may be displayed as a border around this shape comprising a color. As discussed above, the color may indicate whether the distal blood pressure is progressing towards the target distal pressure value (e.g., green color), remaining constant and/or moving away from the target distal pressure value (e.g., yellow color), whether the distal blood pressure is at the minimum value (e.g., red color), or whether there is insufficient data to make a determination (e.g., black color).

To determine whether the interim set point is progressing, constant, or moving away from the target distal pressure, the controller 104 and/or controller 106 may be configured to compare the current interim set point and the interim set point at a prior time point. If the difference between the current interim set point and the interim set point at a prior time point is greater than or equal to a progress value, then the user interface may display the first indicator (e.g., green rectangle). In some variations, the prior time point may be three minutes, four minutes, or five minutes prior to the current time point. In variations in which the prior time point is five minutes prior, the progress value may be 2 mmHg, meaning it has incremented two out of a possible five times over the period between the current time point and the prior time point (e.g., a five minute time interval/window). Therefore, if the difference between the interim set point at a current time point and the interim set point at the prior time point is greater than or equal to 2 mmHg, the distal blood pressure may be progressing towards the target distal pressure value. In some variations, the progress value may be between about 1 mmHg and about 5 mmHg (including all values and sub-ranges therein).

Therefore, if the difference between the interim set point at a current time point and the interim set point, for examples, five minutes ago is greater than or equal to 2 mmHg, the distal blood pressure may be progressing towards the target distal pressure value.

In some variations, assessment of forward progress (i.e., increasing of the interim set point) may utilize a different time interval (difference between the current time point and prior time point) than assessment of backward progress (i.e., decreasing of the interim set point). For example, in some variations, assessment of forward progress may utilize a three to five minute time interval, where assessment of backward progress may utilize a one minute time interval. In other words, in some variations, over five minutes, the difference between the current and prior interim distal set points can be no greater than five, but can be a strongly negative number. A value of zero may reflect no progress, whereas negative numbers may reflect negative progress or put differently, downward incrementing of the interim goal. This can occur rapidly, within a one minute time interval. As such, determinations regarding stability versus instability may be asymmetric with respect to time. Accordingly, in some variations, the controller may utilize a minimum sampling window of three to five minutes to determine stability, but can determining stability within one minute.

Additionally or alternatively, if the current interim set point is equal to the target distal pressure value, then the user interface may display the first indicator (e.g., green).

If the interim set point during the prior window is equal to the target distal pressure and the difference between the current interim set point and the target distal pressure value is less than the indicator value, the user interface may display the second indicator (e.g., yellow color). As discussed above, in some variations, the indicator value may be −1 mmHg. That is, if the interim set point over the last minute, last two minutes, last three minutes, last four minutes, and/or the last five minutes is equal to the target distal pressure value and if the current interim set point is more than 1 mmHg away from the target distal pressure value, the distal blood pressure may be constant or moving away from the target distal pressure value.

Furthermore, if the difference between the current interim set point and the interim set point at a prior time point is less than the progress value, the user interface may display the second indicator (e.g., yellow color). In some variations, as discussed above, the prior time point may be five minutes prior to the current time point. In some variations, as discussed above, the progress value may be 2 mmHg. Therefore, in these variations, if the difference between the interim set point at a current time point and the interim set point five minutes ago is less than 2 mmHg, the distal blood pressure may be constant and/or moving away from the target distal pressure value. As described above, in some variations, the progress value for display of the first indicator may be any value above a specific setpoint in a range of 1 mmHg to 5 mmHg, whereas display of the second indicator may occur at any progress value below this specific setpoint.

If the distal blood pressure value is at a minimum value, the user interface may display the third indicator (e.g., red). In some variations, inflation of the expandable member 110 may impede the blood flow beyond the expandable member. Accordingly, the distal blood pressure may descend to a minimum value (e.g., to a minimum value when the blood vessel is fully occluded). Therefore, the third indicator may indicate that the blood flow proximal to the expandable member 110 may be impeded, thereby indicating that the expandable member may be at occlusion or may be near occlusion.

If the controller 104 and/or controller 160 does not have enough information to determine the progress of the distal blood pressure toward the distal target pressure, the user interface may display a fourth indicator. As an example, if the user switches from an automatic mode of operation to a manual mode of operation, or vice versa, the controller 104 and/or controller 106 may not have enough data to determine the progress of the distal blood pressure. In such scenarios, the user interface may display the fourth indicator (e.g., black, gray).

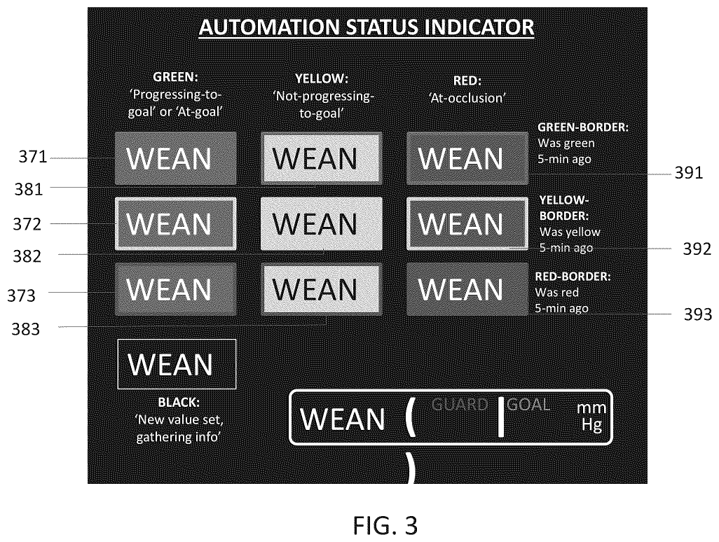

FIG. 3 illustrates an exemplary variation of a user interface displaying progress of the distal blood pressure at a current time point and at a prior point in time. In FIG. 3, the controller 104 and/or controller 106 may be in the wean to off control state. Although, not displayed in FIG. 3, it should be readily understood that the user interface may similarly display the progress of the distal blood pressure when the controller 104 and/or the controller 106 is in a distal pressure control state. The progress at a current time point may be displayed as a rectangular box comprising a color. The progress at a prior point in time may be displayed as a border around the rectangular box. The user interface may display a green color if the interim set point is progressing towards a target distal pressure value, a yellow color if the interim set point is constant and/or moving away from the target distal pressure value, and red color if the distal pressure value is at a minimum possible value.

In FIG. 3, rectangular box 371 may indicate that the interim set point is progressing towards the target distal pressure value at a current time point and at a prior point in time (e.g., five minutes ago). However, rectangular box 372 may indicate that although the interim set point is progressing towards the target distal pressure value at a current time point, the interim set point was constant or is moving away from the target distal pressure value at the prior point in time (e.g., five minutes ago). Rectangular box 373 may indicate that the interim set point may be progressing towards the target distal pressure value at a current time point and that the distal pressure value was at a minimum value at the prior point in time. Similarly, rectangular box 381 may indicate that while the interim set point is constant and/or moving away from the target distal pressure value at a current time point, the interim set point was progressing towards the target distal pressure value at the prior point in time (e.g., five minutes ago). Rectangular box 382 may indicate that the interim set point is constant and/or moving away from the target distal pressure value at a current time point and at the prior point in time. Rectangular box 383 may indicate that the interim set point may be constant and/or moving away from the target distal pressure value at a current time point and that the distal pressure value was at a minimum value at the prior point in time. In this context, if the prior time point reflected the minimum value (third indicator value), but now the current interim set point is moving away from the target distal pressure value, this may indicate that the distal pressure trigger for the third indicator value (reflecting occlusion or near occlusion) has decreased, enabling further inflation/expansion of the expandable member and a corresponding decrease in the interim setpoint.

Similarly, rectangular box 391 may indicate that distal pressure value is at a minimum at the current point in time but that the interim set point was progressing towards the target distal pressure value at the prior point in time. Rectangular box 392 may indicate that distal pressure value is at a minimum at the current point in time but that the interim set point was constant and/or moving away from the target distal pressure value at the prior point in time. Rectangular box 393 may indicate that the distal pressure value is at a minimum at the current point in time and the prior point in time.

### METHOD OF AUTOMATICALLY CONTROLLING AN EXPANDABLE MEMBER

Generally, methods for automatically controlling an expandable member may include obtaining proximal sensor data from a proximal pressure sensor and distal sensor data from a distal pressure sensor, comparing the obtained proximal sensor data to a proximal pressure threshold value, adjusting a volume of the expandable member based on the distal sensor data in response to determining that the proximal sensor data is above or at the proximal pressure threshold value, and adjusting the volume of the expandable member based on the proximal sensor data in response to determining that the proximal sensor data is below the proximal pressure threshold value.

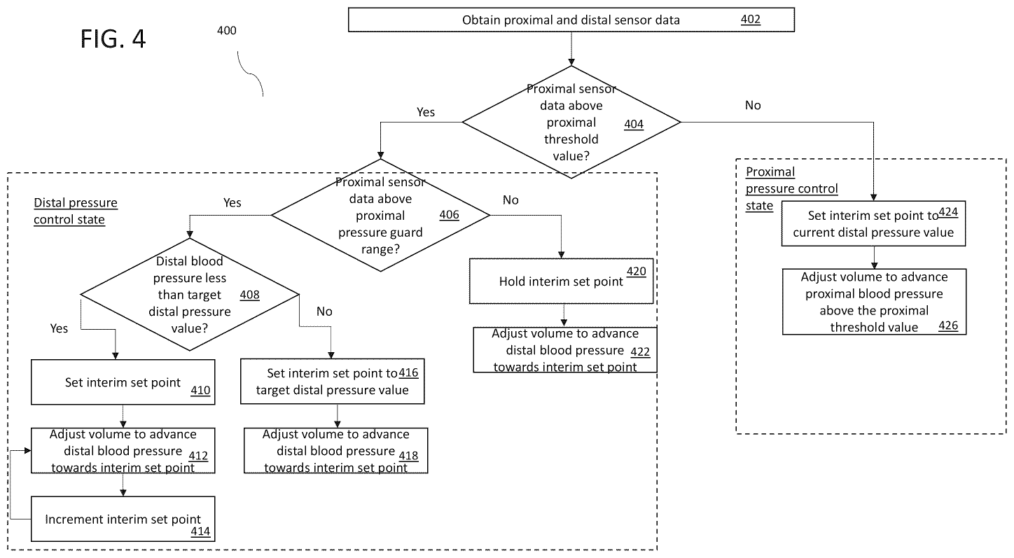

FIG. 4 is a flowchart illustrating an exemplary variation of a method 400 for automatically controlling an expandable member. In some variations, the method 400 may include advancing an expandable member to and within a blood vessel of a subject. A proximal pressure sensor may be positioned distal to the expandable member and a distal pressure sensor may be positioned proximal to the expandable member. A controller may be operably and/or communicatively coupled to the expandable member. A user may choose to operate the controller in an automatic mode of operation. In the automatic mode of operation, the controller may autonomously adjust a volume of the expandable member.

For example, at 402, the method 400 may include obtaining proximal sensor data from the proximal pressure sensor and distal sensor data from distal pressure sensor. The proximal sensor data may include proximal blood pressure such as systolic proximal blood pressure, diastolic proximal blood pressure, PMAP, a combination thereof, and/or the like. The distal sensor data may include distal blood pressure such as systolic distal blood pressure, diastolic distal blood pressure, DMAP, a combination thereof, and/or the like.

At 404, the method may include comparing the proximal sensor data to a proximal pressure threshold value. For instance, the method 400 may include comparing proximal blood pressure (e.g., PMAP) to the proximal pressure threshold value. In some variations, the method 400 may include receiving, via a user interface, the proximal pressure threshold value from a user. In some variations, the method 400 may include determining the proximal pressure threshold value for the subject automatically using the controller. For example, the method may include determining proximal pressure threshold value for a subject based on the physiologic conditions of the subject.

If the proximal sensor data is above the proximal pressure threshold value, the method 400 may include transitioning the controller to a distal pressure control state. At 406, the method 400 may include comparing the proximal sensor data to a proximal pressure guard range. A lower limit of the proximal pressure guard range may be the proximal pressure threshold value. The upper limit of the proximal pressure guard range may be a predetermined amount of pressure above the proximal pressure threshold value. Therefore, the upper limit of the proximal pressure guard range may be the proximal pressure threshold value plus the predetermined amount. In some variations, the predetermined amount may be 5 mmHg.

If the proximal sensor data is above the upper limit of the proximal pressure threshold value, the method 400 may include transitioning the controller to an adjust state. In the adjust state, the method 400 may include at 408, comparing the distal sensor data to a target distal pressure value. For example, the method may include comparing DMAP to a target distal pressure value. In some variations, the target distal pressure value may be received via a user interface from a user before or during an endovascular procedure.

If the distal sensor data is less than the target distal pressure value, then at 410, the method 400 may include setting an interim set point. If the controller is transitioned to the adjust state immediately after initial occlusion (e.g., immediately after the expandable member reaches occlusion for a first time), the method 400 may include setting the interim set point to a predetermined interim value immediately after occlusion. The predetermined interim value may be 5 mmHg. At 412, the method may include adjusting a volume of the expandable member so that the distal sensor data approaches the interim set point. After adjusting the volume of the expandable member, at 414 the method may include incrementing the interim set point. As discussed above, the interim set point may be incremented multiple times. For example, the interim set point may be a stepwise function. In some variations, 400 may include incrementing the interim set point by a predetermined value after a set period of time. For instance, the method may include incrementing the interim set point by 1 mmHg after every minute. After incrementing the interim set point, the method 400 may include adjusting the volume of the expandable member so that the distal sensor data approaches the interim set point each time the interim set point is updated (e.g., incremented). For example, the volume of the expandable member 110 may be adjusted every couple of seconds (e.g., every 6 seconds) so that the distal blood pressure approaches the interim set point.

In some variations, the method 400 may include additionally monitoring the progress of the distal sensor data towards the interim set point. For instance, the method 400 may include comparing a difference between the distal sensor data and the interim set point to a lagging range. If the difference is within the lagging range, the method may include incrementing the interim set point by the predetermined value after the set period of time (e.g., as discussed at 414). However, if the difference is outside (e.g., below) the lagging range, then the method 400 may include holding the interim set point constant until the distal sensor data advances closer to the interim set point.

If the distal sensor data is more than the target distal pressure value, then at 416, the method 400 may include setting the interim set point to the target distal pressure value. At 418, the method may include adjusting the volume of the expandable member so as to advance the distal sensor data towards the interim set point.

If, however, at 406, the proximal sensor data is within the proximal pressure guard range, the method 400 may include transitioning the controller to a hold state. In the hold state, the method 400 may include at 420, holding the interim set point constant. At 422, the method 400 may include adjusting the volume of the expandable member so as to advance the distal sensor data towards the (constant) interim set point.

The steps above were described for when the proximal sensor data is above the threshold value (e.g., at 404). If, however, at 404, the proximal sensor data is below the proximal threshold value, then the method 400 may include transitioning the controller to a proximal pressure control state. At 424, the method may include setting the interim set point to the current distal pressure value. At 426, the method 400 may include adjusting the volume of the expandable member to advance the proximal blood pressure above the proximal pressure threshold value. Therefore, during an endovascular procedure, the method 400 may include continuously monitoring the proximal blood pressure to the proximal pressure threshold value. The method 400 may include transitioning to the distal pressure control state or the proximal pressure control state based on this comparison. In this manner, the method 400 may control a volume of the expandable member continuously and automatically.

In some variations, towards the end of the endovascular procedure (e.g., prior to removing the expandable member from the subject's blood vessel), the method 400 may include receiving from a user (e.g., via a user interface) an indication that the user wishes to fully deflate the expandable member. In response to the indication from the user to fully deflate the expandable member, the method 400 may include transitioning to a wean to off control state. In the wean to off control state, the method may include setting a target expandable member pressure value to a minimum expandable member pressure value (e.g., when the expandable member is fully deflated). In some variations, the expandable member pressure value may be a negative pressure value. The method 400 may include adjusting the volume of the expandable member to such that the distal blood pressure advances to the target distal pressure value quickly and/or advances to the target distal pressure value at a rate determined by how close the proximal pressure is to the proximal pressure target. In some variations, the advancement towards the distal target values and or interim distal values may be predicated on maintaining at least a minimum proximal pressure value as set by the user and/or as determined by the device. In some variations, the method may include switching from the wean to off mode to a proximal pressure target mode if the proximal pressure drops below the proximal target pressure.

In addition to automatically controlling an expandable member, the methods may include delivering one or more blood products to a patient to help stabilize their condition. Exemplary blood products include without limitation, red blood cells, plasma, platelets, and cryoprecipitate. Other fluids, e.g., normal saline, Ringer's lactate, D5W, or medications may also be infused to assist in obtaining or maintaining hemodynamic stability.

Delivery of the blood products by the devices and/or systems described herein may be manual or automated. When delivered manually, a user may press one or more areas or buttons of a user interface of a controller (e.g., controller 104, controller 106, or a different controller) to stop or start the infusion of the blood product. When delivered automatically, target physiologic conditions for the patient may be input to the controller (e.g., controller 104, controller 106, or a different controller) by a user (e.g., prior to the endovascular procedure and/or during the endovascular procedure). Upon receiving sensor data from one or more sensors (e.g., proximal pressure sensor 111a and distal pressure sensor 111b), the controller may automatically or autonomously start and stop the infusion of the blood product based on the sensor data, target physiologic conditions, and one or more transfusion algorithms.

In some variations, the method may include the step of determining, by the controller, to deliver a blood product based on one or more blood pressure parameters (e.g., a proximal blood pressure, a distal blood pressure) while using the expandable member of the devices described herein to control hemorrhage, and in which transfusion of a blood product may help maintain hemodynamic stability and perfusion of organs proximal and distal to the expandable member.

Selection of a transfusion protocol by the controller may be based on, for example, whether the distal blood pressure is meeting an interim set point within a predetermined period of time on the way to a target distal blood pressure value, where the proximal blood pressure or distal blood pressure values are falling while the expandable member volume remains constant over the predetermined period of time. In other examples, selection of the transfusion protocol by the controller may be based on whether the frequency of expandable member inflations to keep the proximal blood pressure within a proximal pressure guard range exceeds a threshold, or when proximal blood pressure continues to decline despite increases in expandable member volume with automated or manual inflations. In some variations, the blood products may be delivered in one or more fixed volumes, at a fixed rate, or in one or more fixed volumes and at a fixed rate. In other variations, the blood products may be delivered in one or more variable volumes, at a variable rate, or in one or more variable volumes and at a variable rate. When a plurality of blood product volumes are delivered (e.g., one, two, three, four, etc.), the volumes may be transfused continuously (e.g., one volume after the other) or iteratively (e.g., separated by a predetermined amount of time). The continuous or iterative delivery of blood volumes may occur until determination by the controller that one or more threshold blood pressure values (e.g., a proximal blood pressure value, a distal blood pressure value) are met.

In some variations, the method for automatically delivering a blood product to a subject may include obtaining, using a controller, sensor data from at least one of a proximal pressure sensor, a distal pressure sensor, and an expandable member sensor, comparing the obtained sensor data to a target value for at least one of a proximal blood pressure, a distal blood pressure, an expandable member pressure, and an interim set point, and in response to determining that the obtained sensor data fails to meet the target value, delivering one or more volumes of the blood product to the subject.

In some variations, the method may also include determining, using the controller, an amount of the blood product to include in the one or more volumes for delivery to the subject. When a plurality of volumes is delivered, the amount and/or the rate of each volume may stay the same (e.g., as described above for fixed volume and/or fixed rate transfusions). In other variations, when a plurality of volumes is delivered, the amount and/or rate of delivery of some of the volumes may be different (e.g., as described above for variable rate and/or variable volume delivery).

### EXAMPLES OF AUTONOMOUS CONTROL

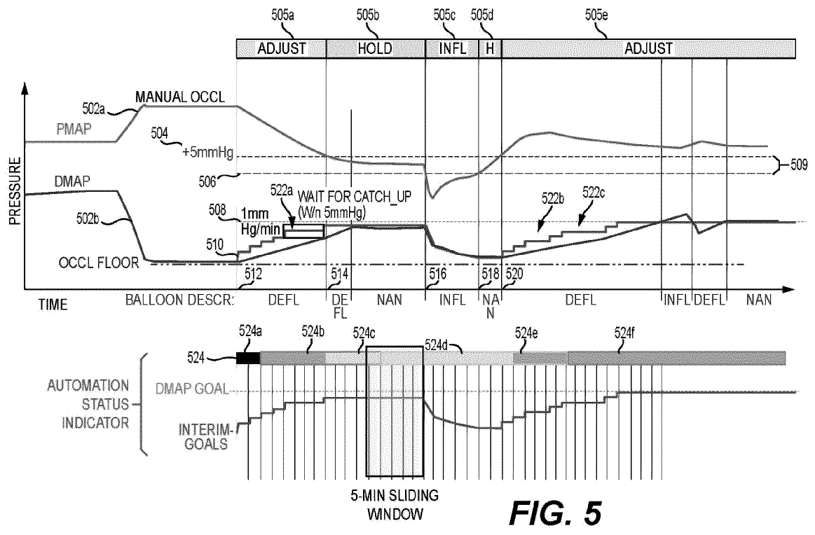

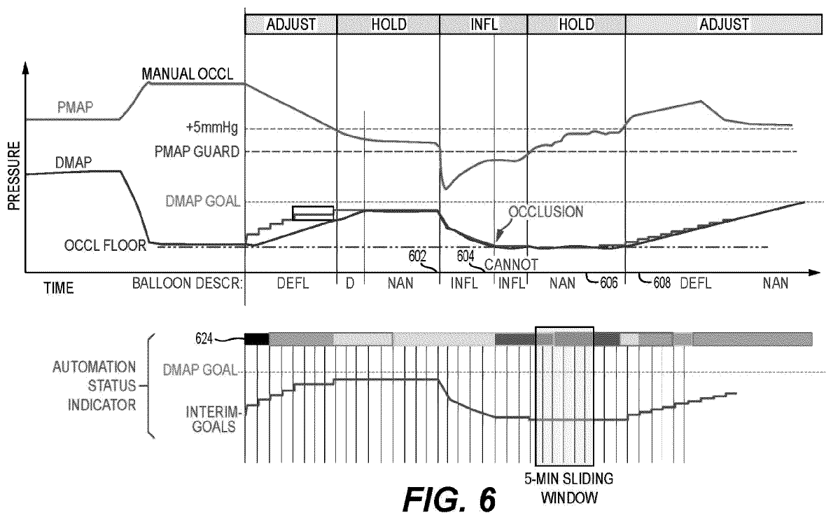

FIG. 5 illustrates an exemplary variation of autonomously controlling an expandable member using the systems, devices, and methods described herein. More specifically, FIG. 5 is a pressure vs. time plot depicting how exemplary sensor data may change over time using the techniques described herein. For example, 502a depicts a change in proximal sensor data (e.g., PMAP) over time and 502b depicts a change in distal sensor data (e.g., DMAP) over time. The proximal pressure threshold value is depicted as PMAP guard 506. The value 504 may be the proximal pressure threshold value 506 plus a buffer amount (e.g., 5 mmHg in FIG. 5). Put differently, the proximal pressure threshold value 506 and the value 504 may represent the lower and the upper boundaries of a pressure range such as the proximal pressure guard range 509 discussed above. As discussed above, the proximal pressure threshold value 506 is the lower limit of the proximal pressure guard range 509. An upper limit of the proximal pressure guard range 509 is depicted as 504. Accordingly, the proximal pressure guard range is between 504 and 506. As discussed above, the controller may transition to a state (e.g., distal pressure control state, such as adjust state, or hold state, and proximal pressure control state) based on a comparison of the proximal sensor data to the proximal pressure guard range 509 or the proximal pressure threshold value 506. These states are depicted in FIG. 5 as 505 (e.g., adjust state 505a, hold state 505b, proximal pressure control state 505c, hold state 505d, and adjust state 505e). In FIG. 5, the target distal pressure value (DMAP GOAL) is represented as 508 and the change in interim set point over time is depicted as 510 (INTERIM GOALS).

In variations in which the volume of the expandable member is adjusted until occlusion, at time point 512, immediately after occlusion, the target distal pressure value is set, or has been set, to a value that is greater than the distal blood pressure. Additionally, at time point 512, the controller may be in a distal pressure state. This is because the proximal blood pressure at time point 512 is greater than the proximal pressure threshold value 506. Additionally, at time point 512, the proximal blood pressure is greater than the proximal pressure guard range 509. Therefore, the controller may be in the adjust state (e.g., adjust state 505a). FIG. 5 also includes an automation status indicator bar 524 that displays progress of the distal blood pressure at a current time point and at a prior point in time.

The controller may set an interim set point. Since time point 512 is immediately after occlusion, the interim set point may be set to a larger predetermined value (e.g., 5 mmHg). The controller may then adjust the volume of the expandable member to advance the distal blood pressure towards the interim set point.

In FIG. 5, the change in the interim set point over time is depicted as 510. As discussed above, the controller may compare the progress of the distal blood pressure with respect to the varying interim set point in the adjust state 505a. For example, the controller may determine a difference between the interim set point and the distal blood pressure. If the difference is within the lagging range (i.e., the distal blood pressure is not lagging behind the interim set point by more than a predetermined lagging value), the interim set point may be incremented, such as, for example, by 1 mmHg every 1 minute as depicted. If the difference is outside the lagging range, then the interim set point may be maintained constant until the distal blood pressure is not lagging behind the interim set point (e.g., catch up operation discussed above). For example, in FIGS. 5, 522a, 522b, and 522c depict periods of time during which the interim set point is held constant in the adjust state. As can be seen in this example, the interim set point may be held constant in the adjust state until the distal blood pressure advances closer to the interim set point.

At 514, the proximal blood pressure descends below the upper limit of the proximal pressure guard range, but remains, until time point 516, above the lower limit of the proximal pressure guard range. Thus, between time points 514 and 516, the proximal blood pressure is within the proximal pressure guard range and thus the controller transitions to a hold state. As seen in FIG. 5, after time point 514, the interim set point is held constant. The volume of the expandable member may be adjusted so that the distal blood pressure approaches the interim set point. The controller continues in the hold state until 516 when the proximal blood pressure descends below the proximal pressure threshold value and thus is outside and below the proximal pressure guard range. Because the proximal pressure has fallen below the proximal pressure threshold value, the controller transitions to a proximal pressure control state. The interim set point at time point 516 is set to be the distal blood pressure at time point 516 and the volume of the expandable member may be adjusted based on the proximal sensor data. In particular, the volume of the expandable member may be adjusted so that the proximal blood pressure ascends above the proximal pressure threshold value and thus re-enters the proximal pressure guard range. After the proximal blood pressure ascends above the proximal pressure threshold value (e.g., at time point 518), the controller transitions to the distal pressure control state, and in particular, the hold state of the distal pressure control state (e.g., since the proximal blood pressure is within the proximal pressure guard range). At time point 520, the proximal blood pressure ascends above the upper limit of the proximal pressure guard range, and thus the controller transitions to the adjust state. In this manner, the volume of the expandable member may be adjusted until the distal blood pressure reaches the target distal pressure value.

Referring now the automation status indicator portion of FIG. 5, status indicator bar 524 depicts the progress of the distal blood pressure towards the target distal pressure value at a current time point and at a prior point in time as it may be depicted, in some variations, on a user interface. For example, the progress at a current time point may be displayed as a rectangular box comprising a color in the status indicator bar 524. The progress at a prior point in time may be displayed as a border around the rectangular box in the status indicator bar 524. The status indicator bar 524 may display a green color if the interim set point is progressing towards a target distal pressure value, a yellow color if the interim set point is constant or moving away from the target distal pressure value, and red color if the distal pressure value is at a minimum possible value. In some variations, the status indicator bar 524 may display black or gray color (or the like) if there is not enough data to determine progress. For instance, immediately after occlusion, for the first two minutes, the controller may not have enough data to determine the progress of the distal blood pressure. In some variations, the status indicator bar 524 may display black or gray color when the controller cannot determine the progress of the distal blood pressure. Accordingly, the user interface may display a first status indicator, depicted in FIG. 5 as a black rectangle. Additionally, because the controller also does not have sufficient data on progress of the distal blood pressure relative to the target distal pressure value for the prior point in time (e.g., 5 minutes ago), the user interface may also display the border around the rectangle with black color. After the first two minutes, the controller may begin determining the progress of the interim set point towards the target distal pressure value. If the interim set point is progressing towards the target distal pressure value, the user interface may display a second, different indicator depicted here as a green rectangle. The border around the rectangle may be indicative of the progress of the interim set point at a prior time, for example, here, at five minutes ago. However, if the interim set point is constant or moving away from the target distal pressure value, the user interface may display a third user interface, displayed here as a yellow rectangle.

FIGS. 6-10 illustrate additional exemplary variations of autonomously controlling an expandable member based on sensor data. Similar to FIG. 5, FIG. 6 is a pressure vs. time plot depicting how exemplary sensor data may change over time using the techniques described herein. Unlike in FIG. 5, FIG. 6 depicts exemplary sensor data when the distal blood pressure descends to a minimum pressure value (e.g., distal blood pressure at occlusion). The status bar indicator 624 in FIG. 6 depicts the status at minimum distal pressure value as further described below. For example, in FIG. 6, at time point 602, the proximal blood pressure may descend below the proximal pressure threshold value and the interim set point may be constant or may move away from the target distal pressure value. Accordingly, the user interface may display a yellow rectangle. The controller may transition to the proximal pressure control state. At 604, however, the distal blood pressure descends to the blood pressure at occlusion. At this point, the blood vessel may be fully occluded.

Accordingly, the user interface may display aa red rectangle. The border around the red rectangle may be displayed with a yellow color, thereby indicating that the interim set point five minutes ago may have been constant or may have been moving away from the target distal pressure value. Additionally, in FIG. 6, the progress of the distal blood pressure may switch rapidly between time point 606 (e.g., from red to yellow at time point 606) and time point 608 (e.g., from yellow to green at time point 608). Therefore, displaying the progress of the distal blood pressure at a prior time point (e.g., 5 minutes ago) may be advantageous since even if the user misses the status at a time point (e.g., due to distraction, requirement to complete other tasks), the user may still be able to identify the transition points since the progress at the prior time point is displayed.

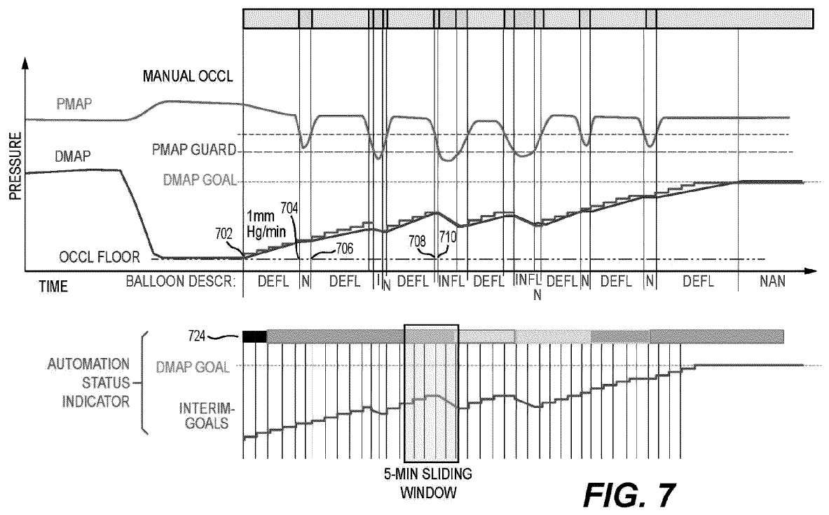

Similar to FIG. 5, FIG. 7 is a pressure vs. time plot depicting how exemplary sensor data may change over time using the techniques described herein. Unlike in FIG. 5, FIG. 7 depicts exemplary sensor data when the proximal blood pressure descends below the upper limit of the proximal pressure guard range and subsequent stabilization of proximal blood pressure before adjusting the volume of the expandable member so that the distal blood pressure continues to advance towards the target distal pressure value. In FIG. 7, the distal blood pressure may be advancing towards the target distal pressure value, such as for example, from time point 702 to 704. During this time period, the controller may be in the adjust state. However, the proximal blood pressure may descend below the upper limit of the proximal pressure guard range (e.g., at time point 704). In such situations, as seen in FIG. 7, the controller may transition to from the adjust state to the hold state to stabilize the proximal blood pressure. Once the proximal blood pressure is stabilized, the controller may transition back to adjust state (e.g., at time point 706) to continue advancing the distal blood pressure towards the target distal pressure value. In some scenarios, after transitioning to the hold state (e.g., at time point 708), the proximal blood pressure may continue to descend to below the proximal pressure threshold value (e.g., at time point 710). In such scenarios, the controller may transition from the distal pressure control state (e.g., the hold state) to the proximal pressure control state (e.g., at time point 710). The status indicator bar 724 depicts the progress of the distal blood pressure towards the target distal pressure value at a current time point and at a prior point in time as it may be depicted, in some variations, on a user interface.

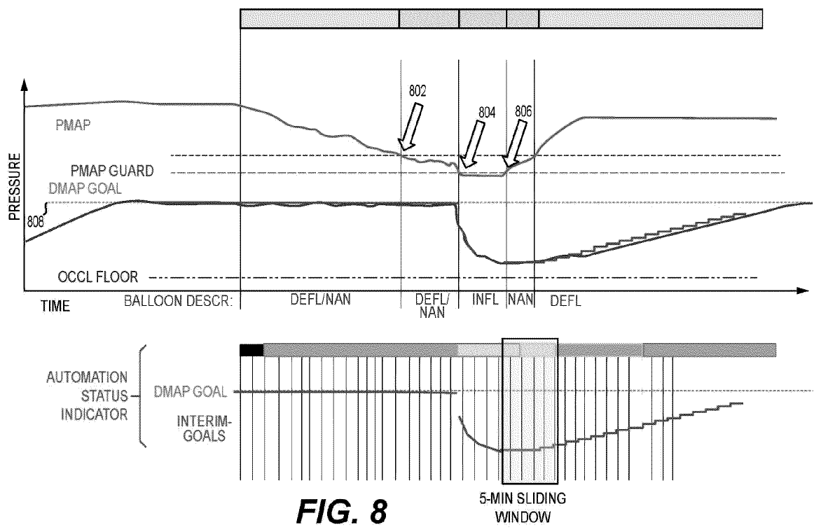

Similar to FIG. 5, FIG. 8 is a pressure vs. time plot depicting how exemplary sensor data may change over time using the techniques described herein. Unlike in FIG. 5, FIG. 8 depicts exemplary sensor data when the proximal blood pressure descends below the proximal pressure threshold value. In FIG. 8, the distal blood pressure may initially be closer to the target distal pressure value 808. That is, the distal blood pressure may be closer and/or at the target distal pressure value 808. However, the proximal blood pressure may descend below the upper limit of the proximal pressure guard range (e.g., at 802). The controller may transition from the adjust state to the hold state of the distal pressure control state. In the hold state, the interim set point may be held constant. At time point 804, the proximal blood pressure may descend below the lower limit of the proximal pressure guard range and the controller may transition to proximal pressure control state. Thus, the controller may re-set the interim set point to the distal blood pressure value and may utilize the proximal sensor data to adjust the volume of the expandable member. At 806, the proximal blood pressure may ascend above the proximal pressure threshold value. Therefore, at 806, the controller may transition from the proximal pressure control state to the distal pressure control state. The controller may continue to vary the interim set point so that the volume of the expandable member is adjusted such that the distal blood pressure advances towards the target distal pressure value.

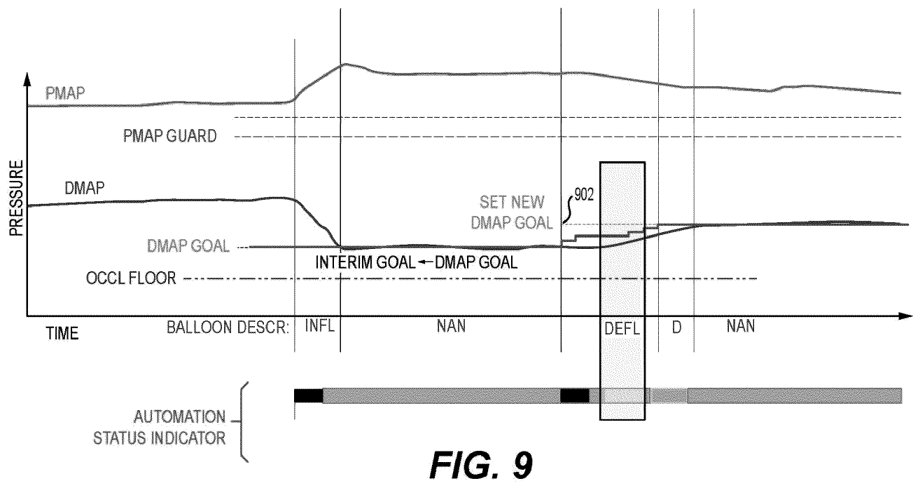

Similar to FIG. 5, FIG. 9 is a pressure vs. time plot depicting how exemplary sensor data may change over time using the techniques described herein. Unlike in FIG. 5, FIG. 9 depicts exemplary sensor data when the distal blood pressure is above the target distal blood pressure value. In FIG. 9, the distal blood pressure may be above the target distal blood pressure value. The controller may be in a distal pressure control state. In such a scenario when the distal blood pressure is above the target distal blood pressure value, the interim set point may be set to the target distal blood pressure value and the volume of the expandable member may be adjusted to move the distal blood pressure to the target distal pressure value. After reaching the target distal pressure value, a user may set a new target distal pressure value (e.g., at time point 902). If the new target distal pressure value is above the distal pressure value at time point 902, the controller may transition back to the adjust state to continue advancing the distal blood pressure towards the new target distal pressure value.

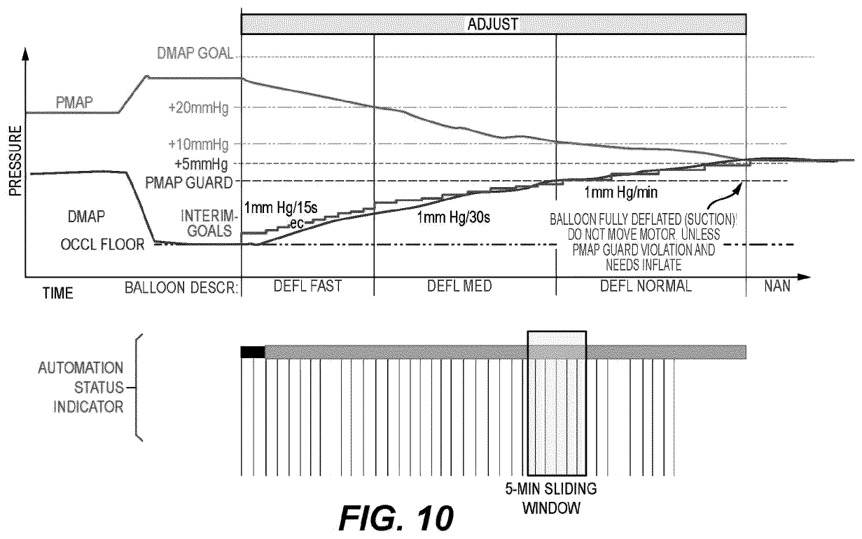

Similar to FIG. 5, FIG. 10 is a pressure vs. time plot depicting how exemplary sensor data may change over time using the techniques described herein. Unlike in FIG. 5, FIG. 10 depicts exemplary sensor data when the controller transitions to a wean off control state. In FIG. 10, the controller may transition to the wean off control state (e.g., per selection by a user). As discussed above, in the wean off control state, the interim set point may be incremented faster than in distal pressure control state. For example, the interim set point may be incremented by 1 mmHg every 15 seconds until the proximal blood pressure is 10 mmHg away from the proximal pressure threshold value. At this point, the interim set point may be incremented in a slower manner (e.g., by 1 mmHg per minute).

The devices, systems and methods described herein may be used to control blood flow in various locations in the body. For example, while described above in relation to use in the aorta, it should be readily understood that the devices, systems, and methods described herein may be used in various vascular procedures such as interventions on arteries such as coronary arteries or cerebral arteries (e.g., thrombectomy procedures for stroke patients, etc.).

### FURTHER EXAMPLE

The following example is illustrative only and should not be construed as limiting the disclosure in any way.

### Fixed Rate, Fixed Volume Blood Product Transfusion

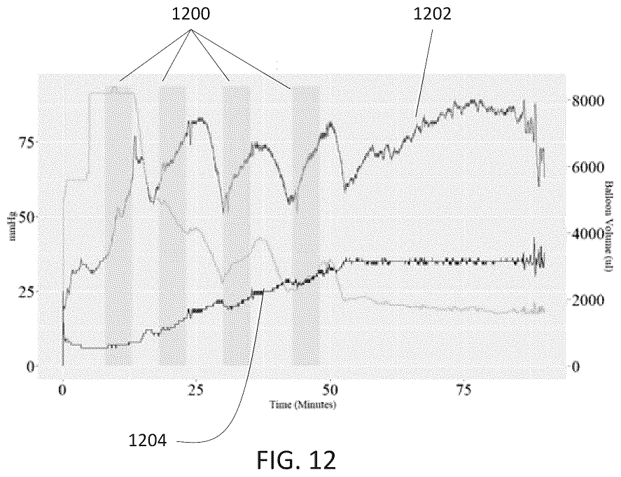

An evaluation of the automated delivery of blood product by the systems and devices described herein according to a fixed rate, fixed volume transfusion was made in a pig model. A catheter comprising an expandable balloon, a proximal blood pressure sensor, and a distal blood pressure sensor, as described herein, was first advanced through the left femoral artery and positioned in the aorta. A liver laceration was then created to induce a controlled hemorrhage, and blood pressure proximal and distal to the balloon measured. The balloon was automatically inflated and deflated according to the Adjust State described above to maintain a target distal blood pressure of 35 mmHg and a proximal pressure value of 60 mmHg. Each time the proximal blood pressure (red line, 1202), as shown in FIG. 12, fell below the proximal pressure value of 60 mmHg for about two minutes, or the interim set point for the target distal pressure did not increase by at least 2.0 mmHg over 5 minutes, the pig was automatically transfused with 3.0 ml/kg of blood at a rate of 3.0 ml/kg over 5 minutes until proximal blood pressure (red line, 1202) reached at least 60 mmHg. After four blood product transfusions (1200), proximal blood pressure (red line, 1202) was maintained above 60 mmHg and distal blood pressure (blue line, 1204) was maintained around the target of 35 mmHg, demonstrating that delivery of blood product at a fixed rate and a fixed volume may help establish hemodynamic stability.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method for automatically controlling an expandable member in a blood vessel of a subject, the method comprising:
obtaining, using a controller, proximal sensor data from a proximal pressure sensor and distal sensor data from a distal pressure sensor;
comparing the obtained proximal sensor data to a proximal pressure threshold value;
in response to determining that the proximal sensor data is above or at the proximal pressure threshold value, adjusting, using the controller, a volume of the expandable member based on the distal sensor data; and
in response to determining that the proximal sensor data is below the proximal pressure threshold value, adjusting, using the controller, the volume of the expandable member based on the proximal sensor data.

2. The method of claim 1, wherein the proximal pressure threshold value is a lower limit of a proximal pressure guard range, and wherein adjusting the volume of the expandable member based on the distal sensor data further comprises:
in response to determining that the proximal sensor data is above an upper limit of the proximal pressure guard range:
adjusting the volume of the expandable member such that the distal sensor data approaches an interim set point, wherein the interim set point is lower than or at a target distal pressure value; and
incrementing, using the controller, the interim set point a plurality of times.

3. The method of claim 2, wherein incrementing the interim set point for a subset of times of the plurality of times comprises for each time of the subset of times incrementing the interim set point by a predetermined value after a set period of time.

4.-5. (canceled)

6. The method of claim 2, wherein incrementing the interim set point comprises for each time of the plurality of times:
comparing, using the controller, a difference between the distal sensor data and the interim set point to a lagging range; and
in response to determining that the difference is within the lagging range, incrementing the set point by a predetermined value after a set period of time.

7. The method of claim 6 further comprising, in response to determining that the difference is below the lagging range, maintaining the interim set point constant.

8. The method of claim 6, wherein the lagging range is between 20 mmHg and 5 mmHg.

9. The method of claim 1, wherein the proximal pressure threshold value is a lower limit of a proximal pressure guard range, and wherein adjusting the volume of the expandable member based on the distal sensor data further comprises:
in response to determining that the proximal sensor data is within the proximal pressure guard range:
holding, using the controller, an interim set point for the distal sensor data constant, wherein the interim set point is lower than or at a target distal pressure value; and
adjusting the volume of the expandable member such that the distal sensor data approaches the interim set point.

10. The method of claim 1, wherein the proximal pressure threshold value is a lower limit of a proximal pressure guard range, and wherein adjusting the volume of the expandable member based on the proximal sensor data further comprises adjusting the volume of the expandable member based on the proximal sensor data until the proximal sensor data is above a lower limit of the proximal guard range.

11. The method of claim 1, wherein adjusting the volume of the expandable member based on the distal sensor data further comprises adjusting the volume of the expandable member so that distal blood pressure obtained from distal sensor data reaches a target distal pressure.

12. The method of claim 11, further comprising indicating whether the distal sensor data is progressing towards the target distal pressure value.

13. The method of claim 12, wherein the indicating comprises:
providing a first indicator if an interim set point is progressing towards the target distal pressure value within a predetermined first time period; and
providing a second, different indicator if an interim set point remains constant or moves away from the target distal pressure value within the predetermined first time period.

14. The method of claim 13, wherein:
providing the first indicator includes determining, using the controller, whether an interim set point during a prior time window is equal to the target distal pressure value and whether a difference between a current interim set point and the target distal pressure value is greater than or equal to an indicator value; and
providing the second indicator includes determining whether the interim set point during the prior time window is equal to the target distal pressure value and whether the difference between the current interim set point and the target distal pressure value is less than the indicator value.

15.-17. (canceled)

18. The method of claim 13, further comprising providing a third indicator providing a progress of the distal sensor data towards the target pressure value during a prior time window.

19.-20. (canceled)

21. The method of claim 13, further comprising:
providing a third indicator to denote that the distal sensor data is at a minimum distal value, wherein the minimum distal value corresponds to the distal sensor data when the blood vessel is fully occluded; and
providing a fourth indicator to denote that a progress of the distal sensor data towards the target distal pressure value cannot be determined by the controller.

22. The method of claim 1 further comprising:
in response to a user input to fully deflate the expandable member, adjusting the volume of the expandable member such that the proximal sensor data remains above the proximal pressure threshold value and the expandable membrane sensor reaches a target expandable member pressure, wherein the target expandable member pressure is a negative pressure.

23.-24. (canceled)

25. The method of claim 1, further comprising automatically delivering a blood product to the subject, wherein the blood product is one or more of red blood cells, plasma, platelets, and cryoprecipitate.

26. (canceled)

27. The method of claim 25, wherein the blood product is delivered in one or more predetermined volumes, at a predetermined rate, or both.

28. The method of claim 25, wherein the blood product is delivered in one or more variably determined volumes, at a variably determined rate, or both.

29.-45. (canceled)

46. A system for controlling blood flow, comprising:
an elongate body comprising an expandable member, a proximal pressure sensor, and a distal pressure sensor;
a pump in fluid communication with the expandable member; and
a controller communicatively coupled to the proximal pressure sensor, the distal pressure sensor, and the pump, the controller configured to:
obtain proximal sensor data from the proximal pressure sensor and distal sensor data from the distal pressure sensor;
compare the obtained proximal sensor data to a proximal pressure threshold value;
in response to determining that the proximal sensor data is above the proximal pressure threshold value, adjust a volume of the expandable member based on the distal sensor data; and
in response to determining that the proximal sensor data is below the proximal pressure threshold value, adjust the volume of the expandable member based on the proximal sensor data.

47. The system of claim 46, wherein the controller is further configured to determine if a blood product is to be delivered in one or more predetermined volumes, at a predetermined rate, or both.

48. The system of claim 46, wherein the controller is further configured to determine if a blood product is to be delivered in one or more variably determined volumes, at a variably determined rate, or both.

49.-59. (canceled)
